(19) United States
(12) Patent Application Publication
(10) Pub. No.: US 2008/0039629 A1
(43) Pub. Date: Feb. 14, 2008

(54) Benzothniazole compositions and their use as ubiquition ligation inhibitors

(75) Inventors: Usha V. Ramesh, Cupertino, CA (US); Rajinder Singh, Belmont, CA (US); Donald G. Payan, Hillsborough, CA (US); Francesco Parlati, San Francisco, CA (US); Raymond Lowe, Castro Valley, CA (US); Gary Charles Look, Santa Clara, CA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: RIGEL PHARMACEUTICALS, INC., South San Francisco, CA (US)

(21) Appl. No.: 11/848,232

(22) Filed: Aug. 30, 2007

Related U.S. Application Data
(62) Division of application No. 10/967,943, filed on Oct. 18, 2004, now abandoned.
(60) Provisional application No. 60/512,034, filed on Oct. 17, 2003.
(60) Provisional application No. 60/609,288, filed on Sep. 13, 2004.

(51) Int. Cl. C07D 215/00; C07D 261/20; C07D 277/82; C07D 417/00; C07D 513/00

(52) U.S. Cl. 546/156; 546/198; 546/270.1; 548/153; 548/159; 548/161

(57) ABSTRACT

This invention describes compounds and pharmaceutical compositions useful as ubiquitin agent inhibitors. The compounds and pharmaceutical compositions of the invention are useful as inhibitors of the biochemical pathways of organisms in which ubiquitination is involved. The invention also comprises the use of the compounds and pharmaceutical compositions of the invention for the treatment of conditions that require inhibition of ubiquitination. Furthermore, the invention comprises methods of inhibiting ubiquitination in a cell comprising contacting a cell in which inhibition of ubiquitination is desired with a pharmaceutical composition according to the invention.

### CROSS REFERENCE TO RELATED APPLICATIONS

This application claims priority from U.S. Provisional Application Ser. No. 60/512,034, filed Oct. 17, 2003, and U.S. Provisional Application Ser. No. 60/609,288, filed Sep. 13, 2004, both of which are incorporated herein by reference in their entirety.

### BACKGROUND OF THE INVENTION

1. Field of the Invention

This invention is in the field of ubiquitin ligation and inhibitors of the ubiquitination pathway. Additionally, this invention is in the field of treating diseases or conditions associated with ubiquitination.

2. Summary of the Related Art

Ubiquitin is a 76 amino acid protein present throughout the eukaryotic kingdom. It is a highly conserved protein and is essentially the identical protein in diverse organisms ranging from humans to yeasts to fruit flies. In eukaryotes, ubiquitin is the key component of the ATP-dependent pathway for protein degradation. Proteins slated for degradation are covalently linked to ubiquitin via an ATP-dependent process catalyzed by three separate enzymes.

Ubiquitin has also been implicated as key components in other biochemical processes. Ubiquitination of the Gag structural protein of Rous Sarcoma virus has been linked to the targeting of Gag to the cell membrane of the host cell where it can assemble into spherical particles and bud from the cell surface. Production of HIV particles has also been associated with ubiquitination and may constitute an important cellular pathway for producing infectious particles. Thus, the ubiquitin pathway may be an important target for treatment of HIV positive patients.

There is a need for inhibitors of ubiquitin ligation that can alter the ATP-dependent ubiquitination of proteins. Inhibition of ubiquitination can regulate the degradation of proteins in ways that assist in treating various disorders. Inhibitors of ubiquitin ligases may also help in treating infectious diseases such as bacterial and viral infections that depend on the cellular biochemical machinery.

The ubiquitination of these target proteins is known to be mediated by the enzymatic activity of three ubiquitin agents. Ubiquitin is first activated in an ATP-dependent manner by a ubiquitin activating agent, for example, an E1. The C-terminus of a ubiquitin forms a high energy thiolester bond with the ubiquitin activating agent. The ubiquitin is then transferred to a ubiquitin conjugating agent, for example, an E2 (also called ubiquitin moiety carrier protein), also linked to this second ubiquitin agent via a thiolester bond. The ubiquitin is finally linked to its target protein (e.g. substrate) to form a terminal isopeptide bond under the guidance of a ubiquitin ligating agent, for example, an E3. In this process, monomers or oligomers of ubiquitin are attached to the target protein. On the target protein, each ubiquitin is covalently ligated to the next ubiquitin through the activity of a ubiquitin ligating agent to form polymers of ubiquitin.

The enzymatic components of the ubiquitination pathway have received considerable attention (for a review, see Weissman, Nature Reviews 2:169-178 (2001)). The members of the E1 ubiquitin activating agents and E2 ubiquitin conjugating agents are structurally related and well characterized enzymes. There are numerous species of E2 ubiquitin conjugating agents, some of which act in preferred pairs with specific E3 ubiquitin ligating agents to confer specificity for different target proteins. While the nomenclature for the E2 ubiquitin conjugating agents is not standardized across species, investigators in the field have addressed this issue and the skilled artisan can readily identify various E2 ubiquitin conjugating agents, as well as species homologues (See Haas and Siepmann, FASEB J. 11:1257-1268 (1997)).

Ubiquitin agents, such as the ubiquitin activating agents, ubiquitin conjugating agents, and ubiquitin ligating agents, are key determinants of the ubiquitin-mediated proteolytic pathway that results in the degradation of targeted proteins and regulation of cellular processes. Consequently, agents that modulate the activity of such ubiquitin agents may be used to upregulate or downregulate specific molecules involved in cellular signal transduction. Disease processes can be treated by such up- or down regulation of signal transducers to enhance or dampen specific cellular responses. This principle has been used in the design of a number of therapeutics, including phosphodiesterase inhibitors for airway disease and vascular insufficiency, kinase inhibitors for malignant transformation and Proteasome inhibitors for inflammatory conditions such as arthritis.

Due to the importance of ubiquitin-mediated proteolysis in cellular process, for example cell cycle regulation, there is a need for a fast and simple means for identifying the physiological role of ubiquitin agents that are catalytic components of this enzymatic pathway, and for identifying which ubiquitin agents are involved in various regulatory pathways. Thus, an object of the present invention is to provide compounds, compositions and methods of assaying for the physiological role of ubiquitin agents, and for providing methods for determining which ubiquitin agents are involved together in a variety of different physiological pathways.

### BRIEF SUMMARY OF THE INVENTION

The invention comprises compounds and pharmaceutical compositions of the compounds for inhibiting ubiquitin agents. The pharmaceutical compositions can be used in treating various conditions where ubiquitination is involved. They can also be used as research tools to study the role of ubiquitin in various natural and pathological processes.

In a first aspect, the invention comprises compounds that inhibit ubiquitination of target proteins.

In a second aspect, the invention comprises a pharmaceutical composition comprising an inhibitor of ubiquitination according to the invention and a pharmaceutically acceptable carrier, excipient, or diluent.

In a third aspect, the invention comprises methods of inhibiting ubiquitination in a cell, comprising contacting a cell in which inhibition of ubiquitination is desired with a pharmaceutical composition comprising a ubiquitin agent inhibitor according to the invention.

In a fourth aspect, the invention provides methods for treating cell proliferative diseases or conditions, comprising administering to a patient in need thereof a pharmaceutical composition comprising an effective amount of a ubiquitin agent inhibitor according to the invention. The invention also provides for the use of a compound or composition of the invention for the manufacture of a medicament for use in treating cell proliferative diseases or conditions.

In a fifth aspect, the invention provides methods for treating HIV infection and related conditions, comprising administering to a patient in need thereof a pharmaceutical composition comprising an effective amount of a ubiquitin agent inhibitor according to the invention. The invention also provides for the use of a compound or composition of the invention for the manufacture of a medicament for use in treating HIV infection and related conditions.

The foregoing only summarizes certain aspects of the invention and is not intended to be limiting in nature. These aspects and other aspects and embodiments are described more fully below. All patent applications and publications of any sort referred to in this specification are hereby incorporated by reference in their entirety. In the event of a discrepancy between the express disclosure of this specification and a patent application or publication incorporated by reference, the express disclosure of this specification shall control.

### DETAILED DESCRIPTION OF THE INVENTION

The invention relates to compounds of the formula: 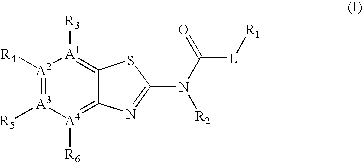
and pharmaceutically acceptable salts thereof, wherein
A¹, A², A³, A⁴ are independently nitrogen or carbon;
L is a bond, —C₁-C₆ alkylene, —C₂-C₆ alkenylene, —NH —, or —NH—C(═O)—;
R₁ is C₁-C₆ alkyl, aryl, heteroaryl, cycloalkyl, heterocyclyl, -aryl-W-aryl, -aryl-W-heterocyclyl, or heterocyclyl-W-aryl, wherein W is a bond, —O—, —SO₂—, or —C(═O)—;
R₂ is H, C₁-C₆ alkyl, or is linked to a carbon of R₁ through a carbonyl group;
R₃ and R₅ are independently H, halogen, or C₁-C₆ alkyl;
R₄ and R₆ are independently H, halogen, C(O)R₇, NR₈R₉, nitro, C₁₋₆-alkyl, C₁₋₆-alkoxy, OCF₃, CF₃, aryl, —C₁₋₆-alkyl-aryl, heteroaryl, —C₁₋₆-alkyl-heteroaryl, C(O)NR₈R₉, C(O)C(O)NR₈R₉, C₁-C₆ alkyl-C(O)—NH—, NR₈R₉—SO₂— or R₁₀—SO₂—; or
   R₃ and R₄ together with the carbon atoms to which they attached form a 5-6 membered aryl or heteroaryl group, wherein the group is optionally substituted with C₁-C₆ alkyl; or
   R₄ and R₅ together with the carbon atoms to which they are attached form a 5-6 membered aryl or heteroaryl group, wherein the group is optionally substituted with C₁-C₆ alkyl;
   provided that if A¹ is nitrogen, R₃ is absent, if A² is nitrogen, R₄ is absent, of A³ is nitrogen, R₅ is absent, and if A⁴ is nitrogen, R₆ is absent;
R₇ is hydrogen, C₁₋₆-alkyl, C₁₋₆-alkoxy, C(Z)-R₁₁ where Z is CH₂ or O, heteroaryl, aryl, or a group of the formula 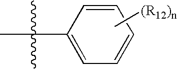
wherein n is 1 to 5 and each R₁₂ is the same or different and is C₁₋₆-alkyl, hydroxy, halogen, nitro, oxo, amino, halo-C₁₋₆-alkyl, C₁₋₆-alkoxy, halo-C₁₋₆-alkoxy, or cyano, NHC(O)—C₁₋₆-alkyl, NHC(O)—C₂₋₆-alkylene, C(O)—O—C₁₋₆-alkyl, or C(O)-aryl;
R₈ and R₉ are independently hydrogen, or C₁-C₆-alkyl;
R₁₀ is C₁₋₆-alkyl, C₁₋₆-alkyl-aryl, aryl, or heteroaryl;
R₁₁ is C₁₋₆-alkyl, C₁₋₆-alkyl-aryl, aryl, or NR₈R₉;
with the proviso that R₄ and R₆ are not simultaneously hydrogen; and
wherein each one of the alkyl, aryl, heteroaryl, or heterocyclyl of R₁ to R₁₂ is optionally substituted with one or more groups selected from C₁₋₈-alkyl, C₂-C₆ alkenyl, hydroxy, halogen, nitro, oxo, amino, monoalkylamino, dialkylamino, halo-C₁₋₈-alkyl, C₁₋₈-alkoxy, halo-C₁₋₈-alkoxy, cyano, NHC(O)—C₁₋₈-alkyl, NHC(O)-cycloalkyl, NHC(O)—C₂₋₆-alkenyl, NHC(O)-aryl-C(O)—O—C₁₋₈-alkyl, C(O)—O—R₁₃, —O—C(O)—C₁-C₈ alkyl, or C(O)-aryl, wherein R₁₃ is H or C₁-C₈ alkyl,
   and two substituents on aryl, together with the atoms to which they are attached, optionally form a dioxane ring.

Preferred compounds of the formula (I) include compounds of formula (II): 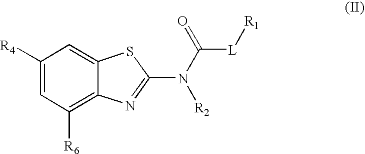
and pharmaceutically acceptable salts thereof, wherein
L is a bond, —C₁-C₆ alkylene-, —C₂-C₆ alkenylene-, —NH—, or —NH—C(═O)—;
R₁ is C₁-C₆ alkyl, aryl, heteroaryl, cycloalkyl, heterocyclyl, -aryl-W-aryl, -aryl-W-heterocyclyl, or heterocyclyl-W-aryl, wherein W is a bond, —O—, —SO₂—, or —C(═O)—;
R₂ is H, C₁-C₆ alkyl, or is linked to a carbon of R₁ through a carbonyl group;
R₄ and R₆ are independently H, halogen, C(O)R₇, NR₈R₉, nitro, C₁₋₆-alkyl, C₁₋₆-alkoxy, OCF₃, CF₃, aryl, —C₁₋₆-alkyl-aryl, heteroaryl, —C₁₋₆-alkyl-heteroaryl, C(O)NR₈R₉, C(O)C(O)NR₈R₉, C₁-C₆ alkyl-C(O)—NH—, NR₈R₉—SO₂— or R₁₀—SO₂—;
   R₇ is hydrogen, C₁₋₆-alkyl, C₁₋₆-alkoxy, C(Z)-R₁₁ where Z is CH₂ or O, heteroaryl, aryl, or a group of the formula 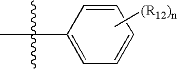
      wherein n is 1 to 5 and each R₁₂ is the same or different and is C₁₋₆-alkyl, hydroxy, halogen, nitro, oxo, amino, halo-C₁₋₆-alkyl, C₁₋₆-alkoxy, halo-C₁₋₆-alkoxy, or cyano, NHC(O)—C₁₋₆-alkyl, NHC(O)—C₂₋₆-alkylene, C(O)O—C₁₋₆-alkyl, or C(O)-aryl;
   R₈ and R₉ are independently hydrogen, or C₁-C₆-alkyl;
   R₁₀ is C₁₋₆-alkyl, C₁₋₆-alkyl-aryl, aryl, or heteroaryl;
   R₁₁ is C₁₋₆-alkyl, C₁₋₆-alkyl-aryl, aryl, or NR₈R₉;
with the proviso that R₄ and R₆ are not simultaneously hydrogen; and
wherein each one of the alkyl, aryl, heteroaryl, or heterocyclyl of the above groups is optionally substituted with one or more groups selected from C₁₋₈-alkyl, C₂-C₆ alkenyl, hydroxy, halogen, nitro, oxo, amino, monoalkylamino, dialkylamino, halo-C₁₋₈-alkyl, C₁₋₈-alkoxy, halo-C₁₋₈-alkoxy, cyano, NHC(O)—C₁₋₈-alkyl, NHC(O)-cycloalkyl, NHC(O)—C₂₋₆-alkenyl, NHC(O)-aryl-C(O)O—C₁₋₈-alkyl, C(O)—O—R₁₃, —O—C(O)—C₁-C₈ alkyl, or C(O)-aryl, wherein R₁₃ is H or C₁-C₈ alkyl,
   and two substituents on aryl, together with the atoms to which they are attached, optionally form a dioxane ring.

Preferred compounds of formula (II) include compounds of formula (II)-1 (and their pharmaceutically acceptable salts), which are compounds of formula (II) in which L is a bond, —NH—, or —NH—C(═O)—, and R₁ is aryl, optionally substituted with one or more groups selected from C₁₋₈-alkyl, C₂-C₆ alkenyl, hydroxy, halogen, nitro, oxo, amino, monoalkylamino, dialkylamino, halo-C₁₋₈-alkyl, C₁₋₈-alkoxy, halo-C₁₋₈-alkoxy, cyano, NHC(O)—C₁₋₈-alkyl, NHC(O)-cycloalkyl, NHC(O)—C₂₋₆-alkenyl, NHC(O)-aryl-C(O)O—C₁₋₈-alkyl, C(O)—O—R₁₃, —O—C(O)—C₁-C₈ alkyl, or C(O)-aryl, wherein R₁₃ is H or C₁-C₈ alkyl, and two substituents on aryl, together with the atoms to which they are attached, optionally form a dioxane ring.

Preferred compounds of formula (II)-1 include those wherein R₁ is phenyl or naphthyl, each of which is optionally substituted with one or two groups selected from C₁₋₈-alkyl, C₂-C₆ alkenyl, hydroxy, halogen, nitro, oxo, amino, monoalkylamino, dialkylamino, halo-C₁₋₈-alkyl, C₁₋₈-alkoxy, halo-C₁₋₈-alkoxy, cyano, NHC(O)—C₁₋₈-alkyl, NHC(O)-cycloalkyl, NHC(O)—C₂₋₆-alkenyl, NHC(O)-aryl-C(O)—O—C₁₋₈-alkyl, C(O)—O—R₁₃, —O—C(O)—C₁-C₈ alkyl, or C(O)-aryl, wherein R₁₃ is H or C₁-C₈ alkyl.

Preferred compounds of formula (II)-1 also include those wherein R₁ is phenyl, optionally substituted with one or two groups selected from C₁₋₈-alkyl, C₂-C₆ alkenyl, hydroxy, halogen, nitro, oxo, amino, monoalkylamino, dialkylamino, halo-C₁₋₈-alkyl, C₁₋₈-alkoxy, halo-C₁₋₈-alkoxy, cyano, NHC(O)—C₁₋₈-alkyl, NHC(O)-cycloalkyl, NHC(O)—C₂₋₆-alkenyl, NHC(O)-aryl C(O)—O—C₁₋₈-alkyl, C(O)O—R₁₃, —O—C(O)—C₁-C₈ alkyl, or C(O)-aryl, wherein R₁₃ is H or C₁-C₈ alkyl.

Preferred compounds of formula (II) further include compounds of formula (II)-2 (and their pharmaceutically acceptable salts), which are compounds of formula (II) wherein L is a bond, —NH—, or —NH—C(═O), and R₁ is heteroaryl, optionally substituted with one or more groups selected from C₁₋₈-alkyl, C₂-C₆ alkenyl, hydroxy, halogen, nitro, oxo, amino, monoalkylamino, dialkylamino, halo C₁₋₈-alkyl, C₁₋₈-alkoxy, halo-C₁₋₈-alkoxy, cyano, NHC(O)—C₁₋₈-alkyl, NHC(O)-cycloalkyl, NHC(O)—C₂₋₆-alkenyl, NHC(O)-aryl-C—(O)—O—C₁₋₈-alkyl, C(O)—O—R₁₃, —O(O)—C₁-C₈ alkyl, or C(O)-aryl, wherein R₁₃ is H or C₁-C₈ alkyl.

Preferred compounds of formula (II) further include compounds of formula (II)-3 (and their pharmaceutically acceptable salts), which are compounds of formula (II) wherein L is a bond, —NH—, or —NH—C(═O)—, and R₁ is cycloalkyl, optionally substituted with one or more groups selected from C₁₋₈-alkyl, C₂-C₆ alkenyl, hydroxy, halogen, nitro, oxo, amino, monoalkylamino, dialkylamino, halo-C₁₋₈-alkyl, C₁₋₈-alkoxy, halo-C₁₋₈-alkoxy, cyano, NHC(O)—C₁₋₈-alkyl, NHC(O)-cycloalkyl, NHC(O)—C₂₋₆-alkenyl, NHC(O)-aryl-C(O)O—C₁₋₈-alkyl, C(O)O—R₁₃, —O—C(O)—C₁-C₈ alkyl, or C(O)-aryl, wherein R₁₃ is H or C₁-C₈ alkyl.

Preferred compounds of formula (II) further include compounds of formula (II)-4 (and their pharmaceutically acceptable salts), which are compounds of formula (II) wherein L is a bond, —NH—, or —NH—C(═O)—, and R₁ is heterocyclyl, optionally substituted with one or more groups selected from C₁₋₈-alkyl, C₂-C₆ alkenyl, hydroxy, halogen, nitro, oxo, amino, monoalkylamino, dialkylamino, halo-C₁₋₈-alkyl, C₁₋₈-alkoxy, halo-C₁₋₈-alkoxy, cyano, NHC(O)—C₁₋₈-alkyl, NHC(O)-cycloalkyl, NHC(O)—C₂₋₆-alkenyl, NHC(O)-aryl-C(O)O—C₁₋₈-alkyl, C(O)—O—R₁₃, —O—C(O)—C₁-C₈ alkyl, or C(O)-aryl, wherein R₁₃ is H or C₁-C₈ alkyl.

Preferred heteroaryl, cycloakyl, and heterocyclyl groups in compounds of formulae (II)-2, (II)-3, and (III)-4 include: pyrolidinyl, indolinyl, indolyl, adamantyl, piperidinyl, cyclohexyl, cyclobutenyl, thiophene, pyridinyl, furanyl, pyrrolyl, thiadiazolyl, benzothiophene, 1,3-dioxoisoindolinyl, pyrazolyl, dihydroquinolinyl, cyclopentyl, and azetidinyl.

Preferred compounds of formulae (II), (II)-1, (II)-2, (II)-3, and (II)-4 include compounds of formula (II)-5 (and their pharmaceutically acceptable salts), which are compounds of formulae (II), (II)-1, (II)-2, (II)-3, or (II)-4 wherein R₆ is hydrogen, and R₄ is C₁₋₆-alkoxy.

Preferred compounds of formula (II)-5 include those wherein R₆ is hydrogen and R₄ is ethoxy or methoxy.

Preferably excluded from the invention is the compound of formula (II) wherein R₄ is methoxy, R₆ is hydrogen, R₂ is hydrogen, L is a bond, and R₁ is benzimidazolyl attached to the main compound at the 2-position of the benzimidazolyl group.

Preferred compounds of the formula (I) include compounds of formula (III): 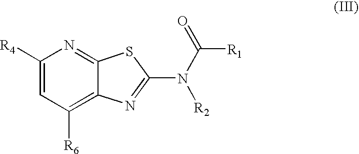
and pharmaceutically acceptable salts thereof, wherein
R₁ is C₁-C₆ alkyl, aryl, heteroaryl, cycloalkyl, heterocyclyl, -aryl-W-aryl, -aryl-W-heterocyclyl, or heterocyclyl-W-aryl, wherein W is a bond, —O—, —SO₂—, or —C(═O)—;
R₂ is H, C₁-C₆ alkyl, or is linked to a carbon of R₁ through a carbonyl group;
R₄ and R₆ are independently H, halogen, C(O)R₇, NR₈R₉, nitro, C₁₋₆-alkyl, C₁₋₆-alkoxy, OCF₃, CF₃, aryl, —C₁₋₆-alkyl-aryl, heteroaryl, —C₁₋₆-alkyl-heteroaryl, C(O)NR₈R₉, C(O)C(O)NR₈R₉, C₁-C₆ alkyl-C(O)—NH—, NR₈R₉—SO₂— or R₁₀—SO₂—;
   R₇ is hydrogen, C₁₋₆-alkyl, C₁₋₆-alkoxy, C(Z)-R₁₁ where Z is CH₂ or O, heteroaryl, aryl, or a group of the formula 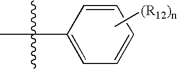
      wherein n is 1 to 5 and each R₁₂ is the same or different and is C₁₋₆-alkyl, hydroxy, halogen, nitro, oxo, amino, halo-C₁₋₆-alkyl, C₁₋₆-alkoxy, halo-C₁₋₆-alkoxy, or cyano, NHC(O)—C₁₋₆-alkyl, NHC(O)—C₂₋₆-alkylene, C(O)O—C₁₋₆-alkyl, or C(O)-aryl;
   R₈ and R₉ are independently hydrogen, or C₁-C₆-alkyl;
   R₁₀ is C₁₋₆-alkyl, C₁₋₆-alkyl-aryl, aryl, or heteroaryl;
   R₁₁ is C₁₋₆-alkyl, C₁₋₆-alkyl-aryl, aryl, or NR₈R₉;
with the proviso that R₄ and R₆ are not simultaneously hydrogen; and
wherein each one of the alkyl, aryl, heteroaryl, or heterocyclyl of the above groups is optionally substituted with one or more groups selected from C₁₋₈-alkyl, C₂-C₆ alkenyl, hydroxy, halogen, nitro, oxo, amino, monoalkylamino, dialkylamino, halo-C₁₋₈-alkyl, C₁₋₈-alkoxy, halo-C₁₋₈-alkoxy, cyano, NHC(O)—C₁₋₈-alkyl, NHC(O)-cycloalkyl, NHC(O)—C₂₋₆-alkenyl, NHC(O)-aryl-C(O)O—C₁₋₈-alkyl, C(O)—O—R₁₃, —O—C(O)—C₁-C₈ alkyl, or C(O)-aryl, wherein R₁₃ is H or C₁-C₈ alkyl,
   and two substituents on aryl, together with the atoms to which they are attached, optionally form a dioxane ring.

Preferred compounds of formula (III) include compounds of formula (III)-1 (and their pharmaceutically acceptable salts), which are compounds of formula III wherein R₁ is aryl, optionally substituted with one or more groups selected from C₁₋₈-alkyl, C₂-C₆-alkenyl, hydroxy, halogen, nitro, oxo, amino, monoalkylamino, dialkylamino, halo-C₁₋₈-alkyl, C₁₋₈-alkoxy, halo-C₁₋₈-alkoxy, cyano, NHC(O)—C₁₋₈-alkyl, NHC(O)-cycloalkyl, NHC(O)—C₂₋₆-alkenyl, NHC(O)-aryl-C(O)—O—C₁₋₈-alkyl, C(O)—O—R₁₃, —O—C(O)—C₁-C₈-alkyl, or C(O)-aryl, wherein R₁₃ is H or C₁-C₈ alkyl, and two substituents on aryl, together with the atoms to which they are attached, optionally form a dioxane ring.

Preferred compounds of formula (III)-1 include compounds wherein R₁ is phenyl, optionally substituted with 1, 2, or 3 groups independently selected from halogen, halo-C₁-C₆ alkyl, cyano, —N—C(O)—C₁-C₆ alkyl, nitro, C₁-C₆ alkoxy, and C₁-C₆ alkyl.

Preferred compounds of formula (III) include compounds of formula (III)-2 (and their pharmaceutically acceptable salts), which are compounds of formula (III) wherein R₁ is heteroaryl, optionally substituted with one or more groups selected from C₁₋₈-alkyl, C₂-C₆ alkenyl, hydroxy, halogen, nitro, oxo, amino, monoalkylamino, dialkylamino, halo-C₁₋₈-alkyl, C₁₋₈ alkoxy, halo-C₁₋₈-alkoxy, cyano, NHC(O)—C₁₋₈-alkyl, NHC(O)-cycloalkyl, NHC(O)—C₂₋₆-alkenyl, NHC(O)-aryl-C(O)—O—C₁₋₈-alkyl, C(O)—O—R₁₃, —O—C(O)—C₁-C₈ alkyl, or C(O)-aryl, wherein R₁₃ is H or C₁-C₈ alkyl.

Preferred compounds of formula (III)-2 include compounds wherein R₁ is thienyl, benzothienyl, furanyl, benzofuranyl, dibenzofuranyl, pyrrolyl, imidazolyl, pyrazolyl, pyridyl, pyrazinyl, pyrimidinyl, indolyl, quinolyl, isoquinolyl, quinoxalinyl, tetrazolyl, oxazolyl, thiazolyl, or isoxazolyl, each of which is optionally substituted with 1, 2, or 3 groups independently selected from halogen, halo-C₁-C₆ alkyl, cyano, —N—C(O)—C₁-C₆ alkyl, nitro, C₁-C₆ alkoxy, and C₁-C₆ alkyl.

Preferred compounds of formula (III)-2 include compounds wherein R₁ is furanyl or thiophene, which are optionally substituted with 1, 2, or 3 groups independently selected from halogen, halo-C₁-C₆ alkyl, cyano, —N—C(O)—C₁-C₆ alkyl, nitro, C₁-C₆ alkoxy, and C₁-C₆ alkyl.

Preferred compounds of formulae (III), (III)-1, and (III)-2 include compounds of formula (III)-3 (and their pharmaceutically acceptable salts), which are compounds of formulae (III), (III)-1, or (III)-2 wherein R₆ is hydrogen, and R₄ is C₁₋₆-alkoxy.

Preferred compounds of formula (III)-3 include those wherein R₆ is hydrogen and R₄ is ethoxy or methoxy.

Preferred compounds of the formula (I) also include compounds of formula (IV): 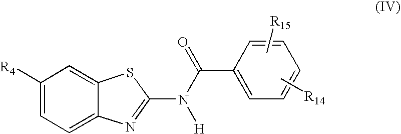
and pharmaceutically acceptable salts thereof, wherein
R₄ is C₁₋₆-alkoxy; and
R₁₄ and R₁₅ are independently H, halogen, amino, nitro, cyano, C₁-C₆ alkyl, C₁-C₆ alkoxy, —C(O)—C₁-C₆ alkyl, —O—C—(O)—C₁-C₆ alkyl, —NH—C(O)—C₁-C₆ alkyl, —NH—C(O)—C₃-C₇ cycloalkyl, —NH—C(O)—C₂-C₆ alkenyl, —SO₂—NR₁₆R₁₇, or
   R₁₄ and R₁₅ together with the atoms to which they are attached form a six membered ring containing one or two heteroatoms atoms selected from —NH— and —O—;
   R₁₆ and R₁₇ are independently H, or C₁-C₆ alkyl,
   or R₁₆ and R₁₇ together with the nitrogen to which they are attached form a 4-8 membered heterocyclic ring, which is optionally substituted.

Preferred compounds of the formula (I) also include compounds of formula (V): 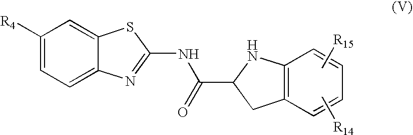
and pharmaceutically acceptable salts thereof, wherein
R₄ is C₁₋₆-alkoxy; and
   R₁₄ and R₁₅ are independently H, halogen, amino, nitro, cyano, C₁-C₆ alkyl, C₁-C₆ alkoxy, —C(O)—C₁-C₆ alkyl, —O—C(O)—C₁-C₆ alkyl, —NH—C(O)—C₁-C₆ alkyl, —NH—C(O)—C₃-C₇ cycloalkyl, —NH—C(O)—C₂-C₆ alkenyl, —SO₂—NR₁₆R₁₇;
      R₁₆ and R₁₇ are independently H, or C₁-C₆ alkyl,
      or R₁₆ and R₁₇ together with the nitrogen to which they are attached form a 4-8 membered heterocyclic ring, which is optionally substituted.

Among preferred compounds of formula (I) are those wherein R₁₂ is C₁₋₆-alkyl, C₁₋₆-alkoxy, halogen, nitro, NHC(O)—C₁₋₆-alkyl, NHC(O)—C₂₋₆-alkylene, C(O)—O—C₁₋₆-alkyl, or C(O)-aryl, R₁ is hydrogen or C₁₋₆-alkyl, and R₃, R₄, R₅ and R₆ are hydrogen, halogen, C₁₋₆-alkoxy, C₁₋₆-alkyl, or nitro.

Other preferred compounds of formula (I) are those wherein R₁₂ is C₁₋₆-alkyl, NHC(O)—C₁₋₆-alkyl, or NHC(O)—C₂₋₆-alkylene, R₄ is C₁₋₆-alkoxy and R₁, R₃, R₅ and R₆ are hydrogen.

Still other preferred compounds of formula (I) are those wherein R₁₂ is methyl, NHC(O)—CH₃, or NHC(O)—(C═CH₂)—CH₃, R₄ is methoxy, and R₁, R₃, R₅, and R₆ are hydrogen.

We have found that the foregoing compounds are useful inhibitors of ubiquitinization, as described more fully below.

Some useful compounds according to one aspect of the invention are given in the following Tables 1 and 2. Compounds in Table 1 are known in the art and commercially available. Compounds in Table 2 can be readily prepared by a person of ordinary skill in the art using the procedures described herein, or by synthetic procedures generally known in the art. Indeed, there is more than one process to prepare the compounds of the invention.

Compounds of the invention include those of formula (I), (II), (II)-1, (II)-2, (II)-3, (II)-4, (II)-5, (III), (III)-1, (III)-2, (III)-3, (IV), and (V), provided that they are not one of the compounds in Table 1.

TABLE 1

| Cmpd | Structure | Name |
|---|---|---|
| 1 | 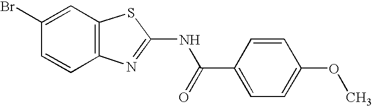 | N-(6-bromo-1,3-benzothiazol-2- yl)-4-methoxybenzamide |
| 3 | 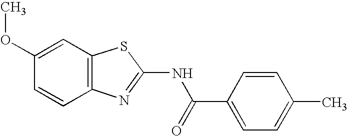 | N-(6-methoxy-1,3-benzothiazoi-2- yl)-4-methylbenzamide |
| 4 | 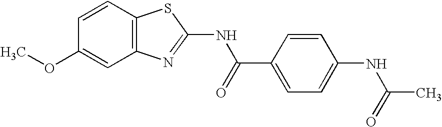 | 4-(acetylamino)-N-(6-methoxy- 1,3-benzothiazol-2-yl)benzamide |
| 5 | 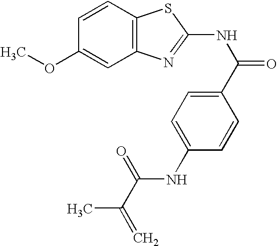 | 4-(methacryloylamino)-N-(6- methoxy-1,3-benzothiazol-2- yl)benzamide |
| 6 | 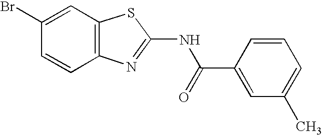 | N-(6-bromo-1,3-benzothiazol-2- yl)-3-methylbenzamide |
| 8 | 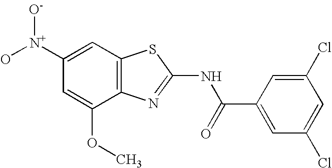 | 3,5-dichloro-N-(4-methoxy-6-nitro- 1,3-benzothiazol-2-yl)benzamide |
| 9 | 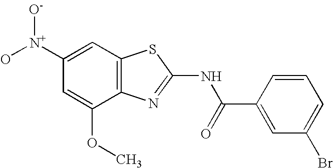 | 3-bromo-N-(4-methoxy-6-nitro- 1,3-benzothiazol-2-yl)benzamide |
| 10 | 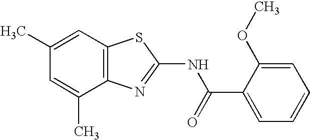 | N-(4,6-dimethyl-1,3-benzothiazol- 2-yl)-2-methoxybenzamide |
| 11 | 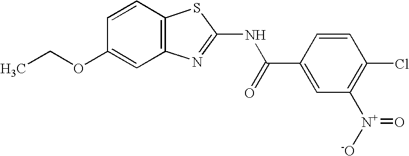 | 4-chloro-N-(6-ethoxy-1,3- benzothiazol-2-yl)-3- nitrobenzamide |
| 12 | 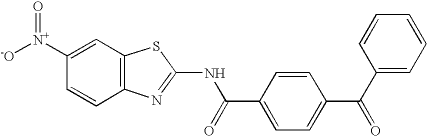 | 4-benzoyl-N-(6-nitro-1,3- benzothiazol-2-yl)benzamide |
| 13 | 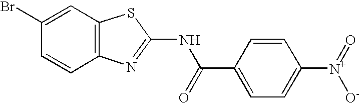 | N-(6-bromo-1,3-benzothiazol-2- yl)-4-nitrobenzamide |
| 14 | 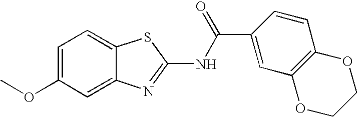 | N-(5-methoxy-1,3-benzothiazol-2- yl)-2,3-dihydro-1,4-benzodioxine- 6-carboxamide |
| 15 | 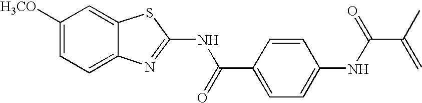 | 4-methacrylamido-N-(6- methoxybenzo[d]thiazol-2- yl)benzamide |
| 16 | 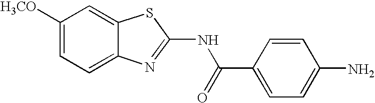 | 4-amino-N-(6- methoxybenzo[d]thiazol-2- yl)benzamide |
| 17 | 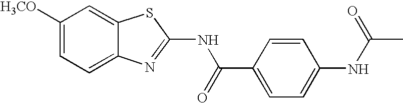 | 4-acetamido-N-(6- methoxybenzo[d]thiazol-2- yl)benzamide |
| 18 | 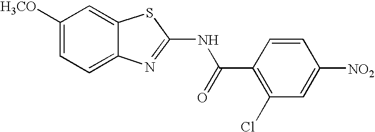 | 2-chloro-N-(6- methoxybenzo[d]thiazol-2-yl)-4- nitrobenzamide |
| 19 | 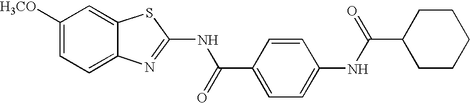 | 4-(cyclohexanecarboxamido)-N- (6-methoxybenzo[d]thiazol-2- yl)benzamide |
| 20 | 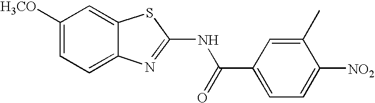 | N-(6-methoxybenzo[d]thiazol-2- yl)-3-methyl-4-nitrobenzamide |
| 21 | 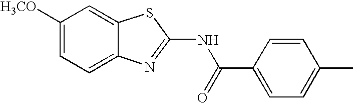 | N-(6-methoxybenzo[d]thiazol-2- yl)-4-methylbenzamide |
| 22 | 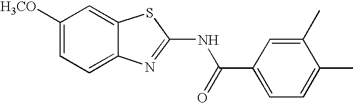 | N-(6-methoxybenzo[d]thiazol-2- yl)-3,4-dimethyl-4-nitrobenzamide |
| 23 | 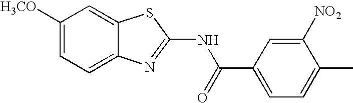 | N-(6-methoxybenzo[d]thiazol-2- yl)-4-methyl-3-nitrobenzamide |
| 24 | 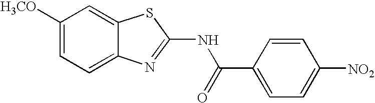 | N-(6-methoxybenzo[d]thiazol-2- yl)-4-nitrobenzamide |
| 25 | 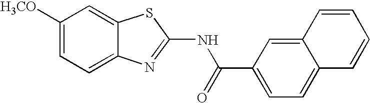 | N-(6-methoxybenzo[d]thiazol-2- yl)-2-naphthamide |
| 26 | 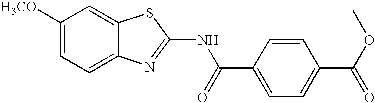 | methyl 4-((6- methoxybenzo[d]thiazol-2- yl)carbamoyl)benzoate |
| 27 | 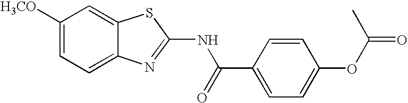 | 4-((6-methoxybenzo[d]thiazol-2- yl)carbamoyl)phenyl acetate |
| 28 | 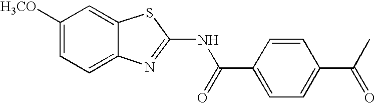 | 4-acetyl-N-(6- methoxybenzo[d]thiazol-2- yl)benzamide |
| 29 | 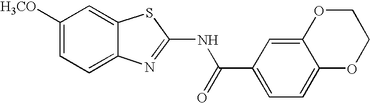 | N-(6-methoxybenzo[d]thiazol-2- yl)-2,3- dihydrobenzo[b][1,4]dioxine-6- carboxamide |
| 30 | 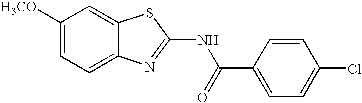 | 4-chloro-N-(6- methoxybenzo[d]thiazol-2- yl)benzamide |
| 31 | 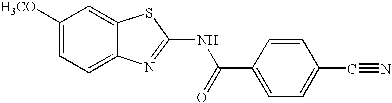 | 4-cyano-N-(6- methoxybenzo[d]thiazol-2- yl)benzamide |
| 32 | 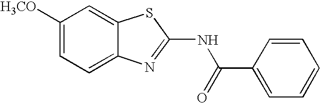 | N-(6-methoxybenzo[d]thiazol-2- yl)benzamide |
| 33 | 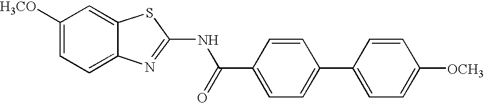 | 4′-methoxy-N-(6-methoxy-1,3- benzothiazol-2-yl)biphenyl-4- carboxamide |
| 34 | 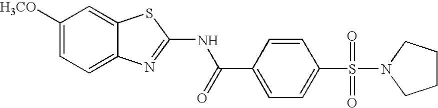 | N-(6-methoxybenzo[d]thiazol-2- yl)-4-(pyrrolidin-1- ylsulfonyl)benzamide |
| 35 | 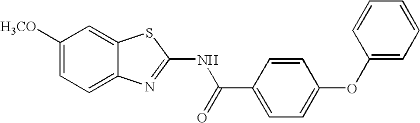 | N-(6-methoxybenzo[d]thiazol-2- yl)-4-phenoxybenzamide |
| 36 | 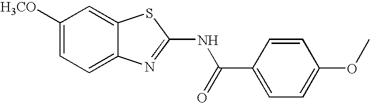 | 4-methoxy-N-(6- methoxybenzo[d]thiazol-2- yl)benzamide |
| 37 | 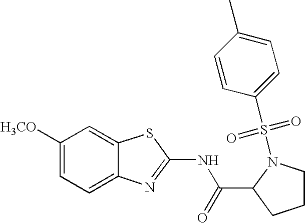 | N-(6-methoxybenzo[d]thiazol-2- yl)-1-tosylpyrrolidine-2- carboxamide |
| 41 | 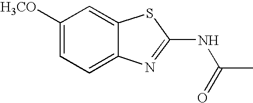 | N-(6-methoxybenzo[d]thiazol-2- yl)acetamide |
| 42 | 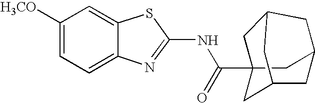 | N-(6-methoxy-1,3-benzothiazol-2- yl)adamantane-1-carboxamide |
| 43 | 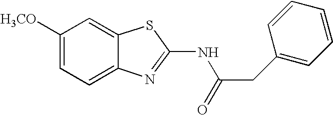 | N-(6-methoxybenzo[d]thiazol-2- yl)-2-phenylacetamide |
| 45 | 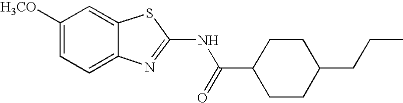 | N-(6-methoxybenzo[d]thiazol-2- yl)-4- propylcyclohexanecarboxamide |
| 49 | 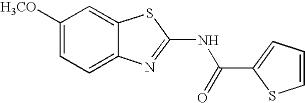 | N-(6-methoxybenzo[d]thiazol-2- yl)thiophene-2-carboxamide |
| 50 | 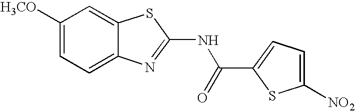 | N-(6-methoxybenzo[d]thiazol-2- yl)-5-nitrothiophene-2- carboxamide |
| 51 | 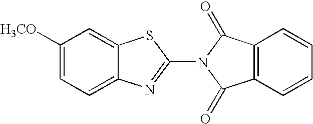 | 2-(6-methoxybenzo[d]thiazol-2- yl)isoindoline-1,3-dione |
| 52 | 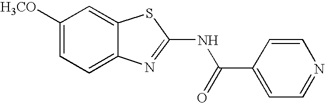 | N-(6-methoxybenzo[d]thiazol-2- yl)isonicotinamide |
| 53 | 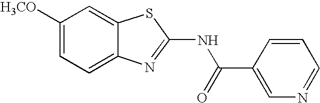 | N-(6-methoxybenzo[d]thiazol-2- yl)nicotinamide |
| 54 | 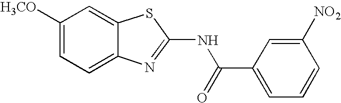 | N-(6-methoxybenzo[d]thiazol-2- yl)-3-nitrobenzamide |
| 55 | 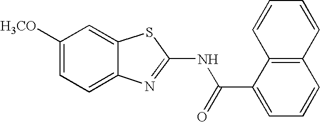 | N-(6-methoxybenzo[d]thiazol-2- yl)-1-naphthamide |
| 57 | 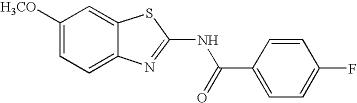 | 4-fluoro-N-(6- methoxybenzo[d]thiazol-2- yl)benzamide |
| 68 | 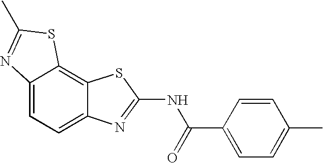 | 3-methyl-N-(7- methyl[1,3]thiazolo[4,5-g][1,3]benzothiazol-2- yl)benzamide |
| 70 | 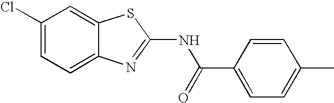 | N-(6-chlorobenzo[d]thiazol-2-yl)-4- methylbenzamide |
| 71 | 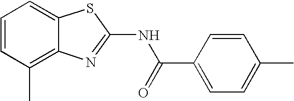 | 4-methyl-N-(4- methylbenzo[d]thiazol-2- yl)benzamide |
| 72 | 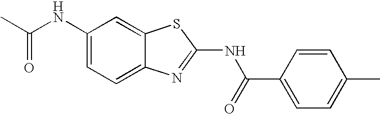 | N-(6-acetamidobenzo[d]thiazol-2- yl)-4-methylbenzamide |
| 73 | 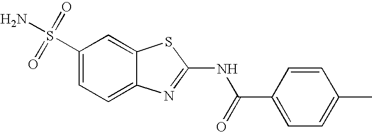 | N-[6-(aminosulfonyl)-1,3- benzothiazol-2-yl]-4- methylbenzamide |
| 74 | 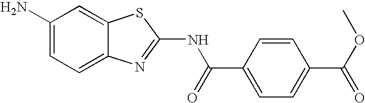 | methyl 4-((6- aminobenzo[d]thiazol-2- yl)carbamoyl)benzoate |
| 76 | 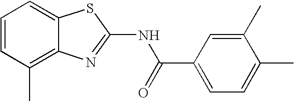 | 3,4-dimethyl-N-(4- methylbenzo[d]thiazol-2- yl)benzamide |
| 77 | 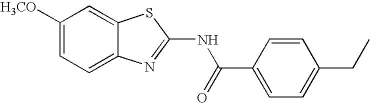 | 4-ethyl-N-(6- methyoxybenzo[d]thiazol-2- yl)benzamide |
| 78 | 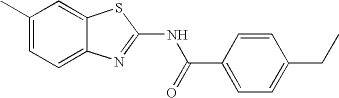 | 4-ethyl-N-(6- methylbenzo[d]thiazol-2- yl)benzamide |
| 79 | 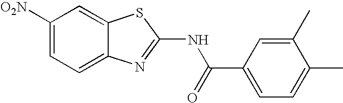 | 3,4-dimethyl-N-(6- nitrobenzo[d]thiazol-2- yl)benzamide |
| 80 | 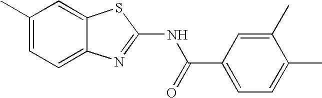 | 3,4-dimethyl-N-(6- methylbenzo[d]thiazol-2- yl)benzamide |
| 81 | 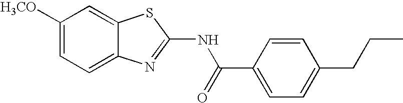 | N-(6-methoxybenzo[d]thiazol-2- yl)-4-propylbenzamide |
| 82 | 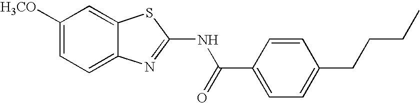 | 4-butyl-N-(6- methoxybenzo[d]thiazol-2- yl)benzamide |
| 83 | 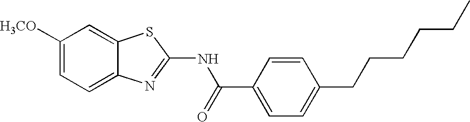 | 4-hexyl-N-(6- methoxybenzo[d]thiazol-2- yl)benzamide |
| 84 | 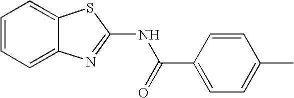 | N-(benzo[d]thiazol-2-yl)-4- methylbenzamide |
| 85 | 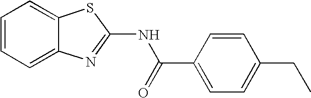 | N-(benzo[d]thiazol-2-yl)-4- ethylbenzamide |
| 86 | 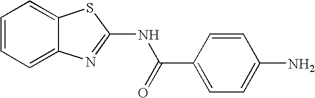 | 4-amino-N-(benzo[d]thiazol-2- yl)benzamide |
| 87 | 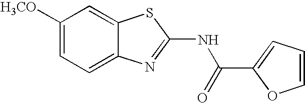 | N-(6-methoxybenzo[d]thiazol-2- yl)furan-2-carboxamide |
| 88 | 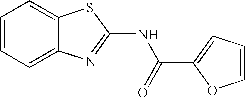 | N-(benzo[d]thiazol-2-yl)furan-2- carboxamide |
| 89 | 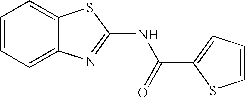 | N-(benzo[d]thiazol-2-yl)thiophene- 2-carboxamide |
| 90 | 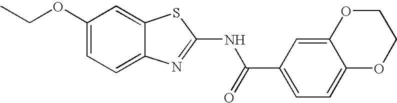 | N-(6-ethoxybenzo[d]thiazol-2-yl)- 2,3-dihydrobenzo[b][1,4]dioxine- 6-carboxamide |
| 91 | 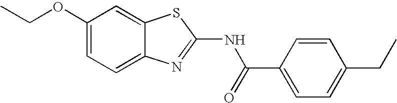 | N-(6-ethoxybenzo[d]thiazol-2-yl)- 4-ethylbenzamide |
| 92 | 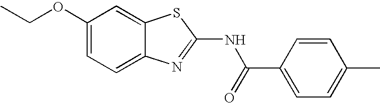 | N-(6-ethoxybenzo[d]thiazol-2-yl)- 4-methylbenzamide |
| 93 | 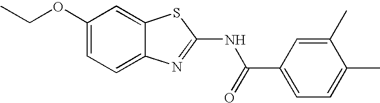 | N-(6-ethoxybenzo[d]thiazol-2-yl)- 3,4-dimethylbenzamide |
| 94 | 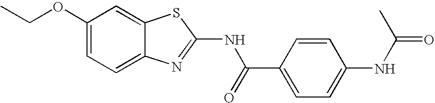 | 4-acetamido-N-(6- ethoxybenzo[d]thiazol-2- yl)benzamide |
| 95 | 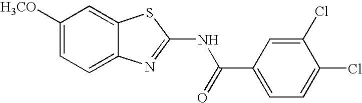 | 3,4-dichloro-N-(6- methoxybenzo[d]thiazol-2- yl)benzamide |
| 96 | 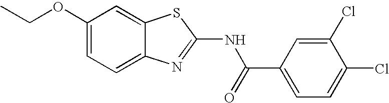 | 3,4-dichloro-N-(6- ethoxybenzo[d]thiazol-2- yl)benzamide |
| 97 | 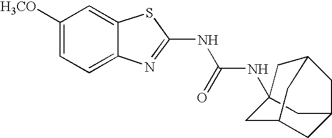 | N-1-adamantyl-N′-(6-methoxy-1,3- benzothiazol-2-yl)urea |
| 98 | 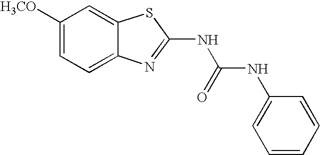 | 1-(6-methoxybenzo[d]thiazol-2-yl)- 3-phenylurea |
| 99 | 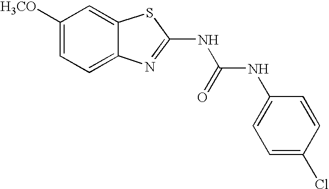 | 1-(4-chlorophenyl)-3-(6- methoxybenzo[d]thiazol-2-yl)urea |
| 100 | 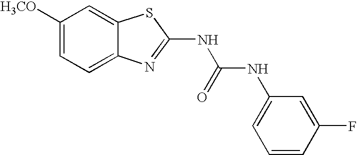 | 1-(3-fluorophenyl)-3-(6- methoxybenzo[d]thiazol-2-yl)urea |
| 102 | 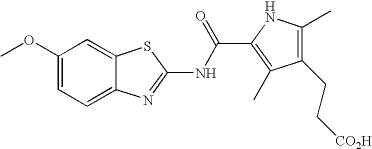 | 3-(5-((6-methoxybenzo[d]thiazol- 2-yl)carbamoyl)-2,4-dimethyl-1H- pyrrol-3-yl)propanoic acid |
| 105 | 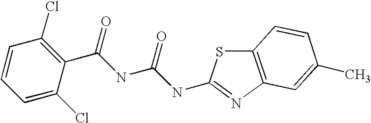 | 1-(2,6-dichlorobenzoyl)-3-(5- methylbenzo[d]thiazol-2-yl)urea |
| 106 | 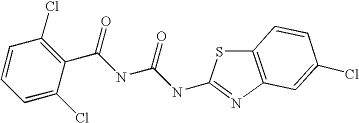 | 1-(5-chlorobenzo[d]thiazol-2-yl)-3- (2,6-dichlorobenzoyl)urea |
| 107 | 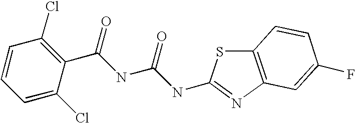 | 1-(2,6-dichlorobenzoyl)-3-(5- fluorobenzo[d]thiazol-2-yl)urea |
| 108 | 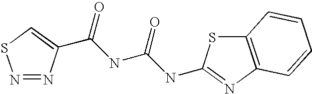 | 1-(benzo[d]thiazol-2-yl)-3-(1,2,3- thiadiazole-4-carbonyl)urea |
| 109 | 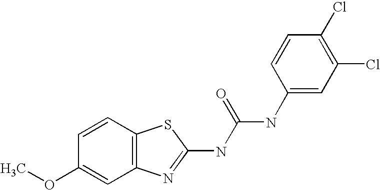 | 1-(3,4-dichlorophenyl)-3-(6- methoxybenzo[d]thiazol-2-yl)urea |
| 110 | 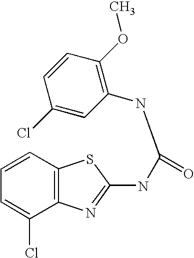 | 1-(5-chloro-2-methoxyphenyl)-3- (4-chlorobenzo[d]thiazol-2-yl)urea |
| 111 | 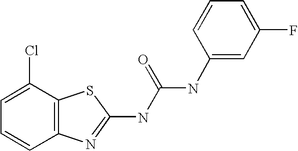 | 1-(4-chlorobenzo[d]thiazol-2-yl)-3- (3-fluorophenyl)urea |
| 112 | 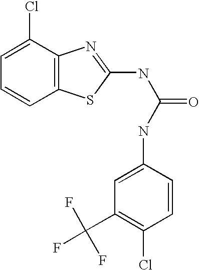 | 1-(4-chloro-3- (trifluoromethyl)phenyl)-3-(4- chlorobenzo[d]thiazol-2-yl)urea |
| 113 | 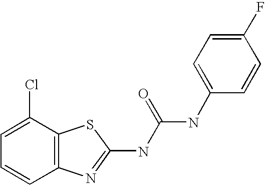 | 1-(4-chlorobenzo[d]thiazol-2-yl)-3- (4-fluorophenyl)urea |
| 114 | 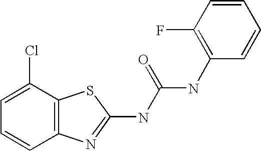 | 1-(4-chlorobenzo[d]thiazol-2-yl)-3- (2-fluorophenyl)urea |
| 115 | 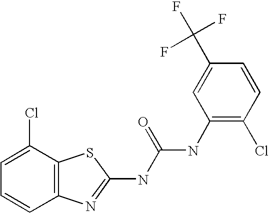 | 1-(2-chloro-5- (trifluoromethyl)phenyl)-3-(4- chlorobenzo[d]thiazol-2-yl)urea |
| 116 | 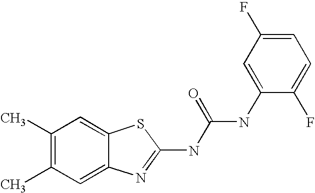 | 1-(2,5-difluorophenyl)-3-(5,6- dimethylbenzo[d]thiazol-2-yl)urea |
| 117 | 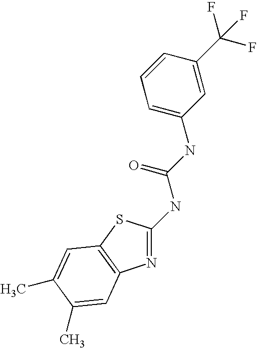 | 1-(5,6-dimethylbenzo[d]thiazol-2- yl)-3-(3- (trifluoromethyl)phenyl)urea |
| 118 | 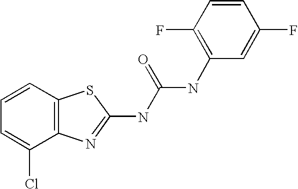 | 1-(7-chlorobenzo[d]thiazol-2-yl)-3- (2,5-difluorophenyl)urea |
| 119 | 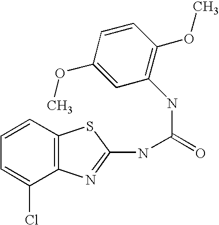 | 1-(4-chlorobenzo[d]thiazol-2-yl)-3- (2,5-dimethoxyphenyl)urea |
| 120 | 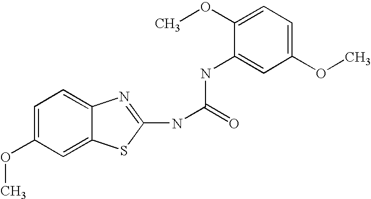 | 1-(2,5-dimethoxyphenyl)-3-(6- methoxybenzo[d]thiazol-2-yl)urea |
| 121 | 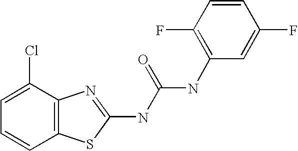 | 1-(4-chlorobenzo[d]thiazol-2-yl)-3- (2,5-difluorophenyl)urea |
| 122 | 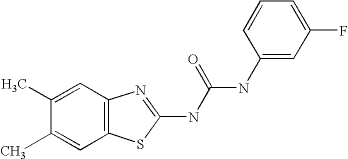 | 1-(5,6-dimethylbenzo[d]thiazol-2- yl)-3-(3-fluorophenyl)urea |
| 123 | 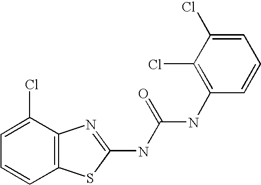 | 1-(4-chlorobenzo[d]thiazol-2-yl)-3- (2,3-difluorophenyl)urea |
| 124 | 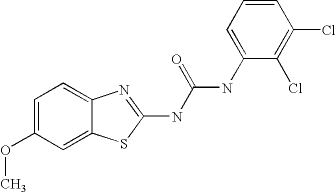 | 1-(2,3-dichlorophenyl)-3-(6- methoxybenzo[d]thiazol-2-yl)urea |
| 125 | 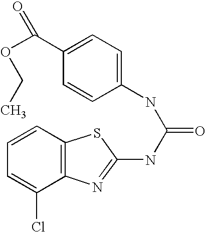 | ethyl (4-(3-(4- chlorobenzo[d]thiazol-2- yl)ureido)benzoate |
| 126 | 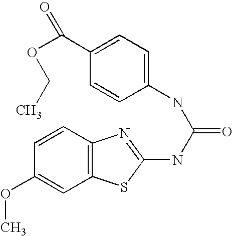 | ethyl (4-(3-(6- methoxybenzo[d]thiazol-2- yl)ureido)benzoate |
| 127 | 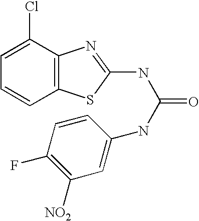 | 1-(4-chlorobenzo[d]thiazol-2-yl)-3- (4-fluoro-3-nitrophenyl)urea |
| 128 | 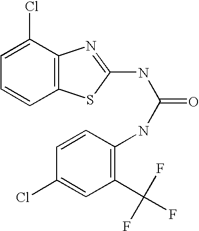 | 1-(4-chloro-2- (trifluoromethyl)phenyl)-3-(4- chlorobenzo[d]thiazol-2-yl)urea |
| 129 | 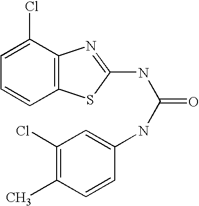 | 1-(3-chloro-4-methylphenyl)-3-(4- chlorobenzo[d]thiazol-2-yl)urea |
| 130 | 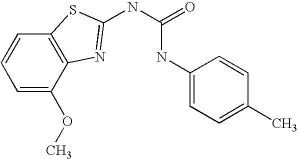 | 1-(4-methoxybenzo[d]thiazol-2-yl)- 3-p-tolylurea |
| 131 | 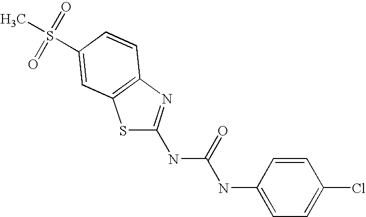 | 1-(4-chlorophenyl)-3-(6- (methylsulfonyl)benzo[d]thiazol-2- yl)urea |
| 132 | 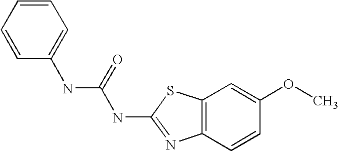 | 1-(6-methoxybenzo[d]thiazol-2-yl)- 3-phenylurea |
| 133 | 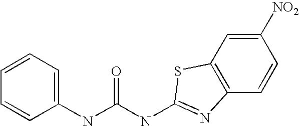 | 1-(6-nitrobenzo[d]thiazol-2-yl)-3- phenylurea |
| 134 | 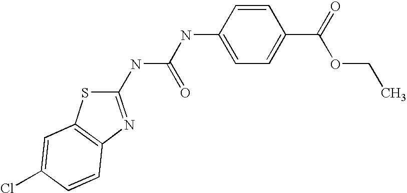 | ethyl 4-(3-(6- chlorobenzo[d]thiazol-2- yl)ureido)benzoate |
| 135 | 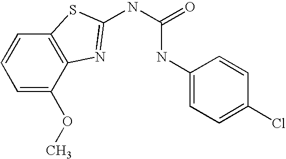 | 1-(4-chlorophenyl)-3-(4- methoxybenzo[d]thiazol-2-yl)urea |
| 136 | 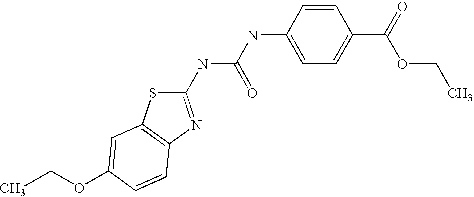 | ethyl 4-(3-(6- ethoxybenzo[d]thiazol-2- yl)ureido)benzoate |
| 139 | 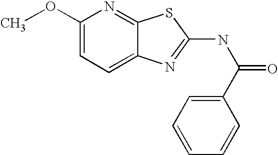 | N-(5-methoxythiazolo[5,4-b]pyridin-2-yl)benzamide |
| 152 | 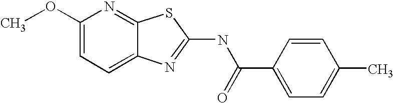 | N-(5-methoxythiazolo[5,4-b]pyridin-2-yl)-4-methylbenzamide |
| 155 | 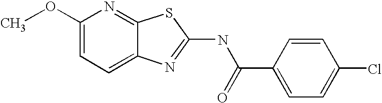 | 4-chloro-N-(5- methoxythiazolo[5,4-b]pyridin-2- yl)benzamide |
| 156 | 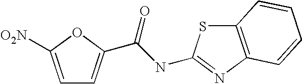 | N-(benzo[d]thiazol-2-yl)-5- nitrofuran-2-carboxamide |
| 157 | 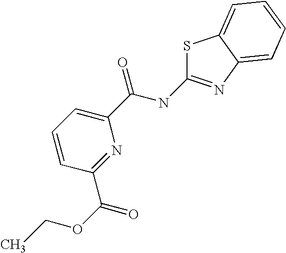 | ethyl 6-(benzo[d]thiazol-2- ylcarbamoyl)picolinate |
| 158 | 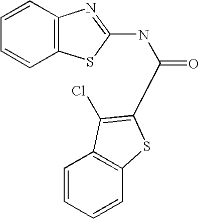 | N-(benzo[d]thiazol-2-yl)-3- chlorobenzo[b]thiophene-2- carboxamide |
| 159 | 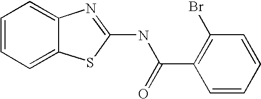 | N-(benzo[d]thiazol-2-yl)-2- bromobenzamide |
| 160 | 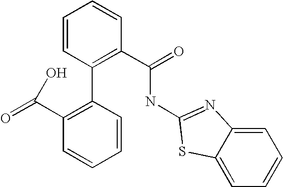 | 2′-[(1,3-benzothiazol-2- ylamino)carbonyl]biphenyl-2- carboxylic acid |
| 161 | 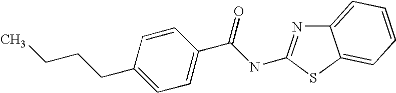 | N-(benzo[d]thiazol-2-yl)-4- butylbenzamide |
| 162 | 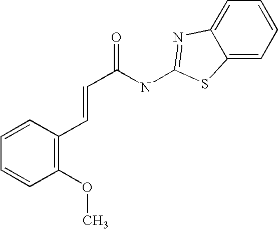 | (E)-N-(benzo[d]thiazol-2-yl)-3-(2- methoxyphenyl)acrylamide |
| 163 | 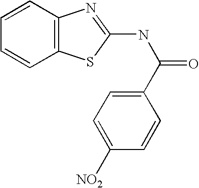 | N-(benzo[d]thiazol-2-yl)-4- nitrobenzamide |
| 164 | 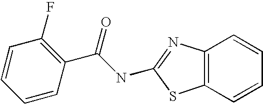 | N-(benzo[d]thiazol-2-yl)-2- fluorobenzamide |
| 165 | 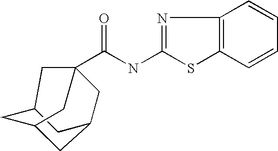 | N-1,3-benzothiazol-2- yladamantane-1-carboxamide |
| 166 | 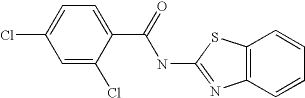 | N-(benzo[d]thiazol-2-yl)-2,4- dichlorobenzamide |
| 167 | 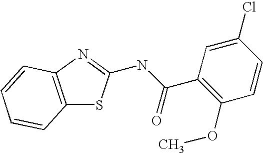 | N-(benzo[d]thiazol-2-yl)-5-chloro- 2-methoxybenzamide |
| 168 | 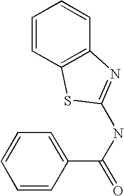 | N-(benzo[d]thiazol-2- yl)benzamide |
| 169 | 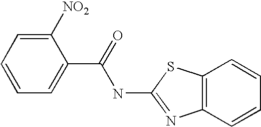 | N-(benzo[d]thiazol-2-yl)-2- nitrobenzamide |
| 170 | 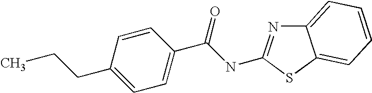 | N-(benzo[d]thiazol-2-yl)-4- propylbenzamide |
| 171 | 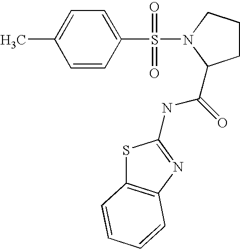 | N-(benzo[d]thiazol-2-yl)-1- tosylpyrrolidine-2-carboxamide |
| 172 | 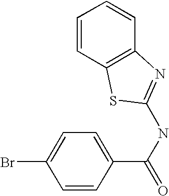 | N-(benzo[d]thiazol-2-yl)-4- bromobenzamide |
| 173 | 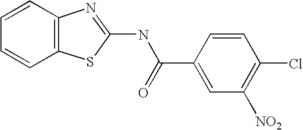 | N-(benzo[d]thiazol-2-yl)-4-chloro- 3-nitrobenzamide |
| 174 | 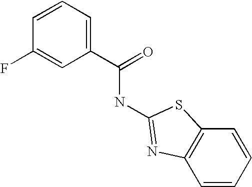 | N-(benzo[d]thiazol-2-yl)-3- fluorobenzamide |
| 175 | 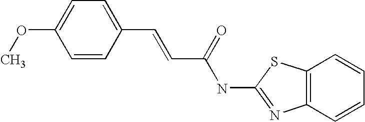 | (E)-N-(benzo[d]thiazol-2-yl)-3-(4- methoxyphenyl)acrylamide |
| 176 | 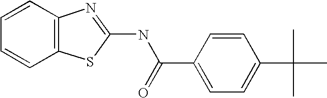 | N-(benzo[d]thiazol-2-yl)-4-tert- butylbenzamide |
| 177 | 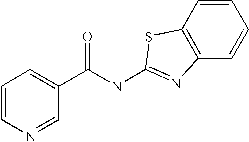 | N-(benzo[d]thiazol-2- yl)nicotinamide |
| 178 | 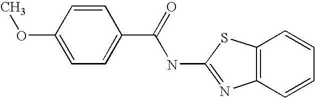 | N-(benzo[d]thiazol-2-yl)-4- methoxybenzamide |
| 179 | 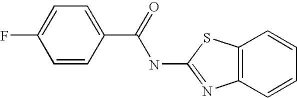 | N-(benzo[d]thiazol-2-yl)-4- fluorobenzamide |
| 180 | 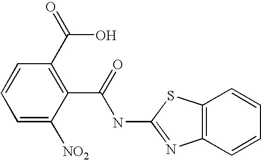 | 2-(benzo[d]thiazol-2- ylcarbamoyl)-3-nitrobenzoic acid |
| 181 | 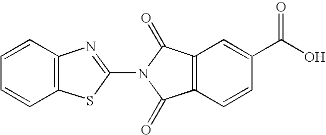 | 2-(benzo[d]thiazol-2-yl)-1,3- dioxosioindoline-5-carboxylic acid |
| 182 | 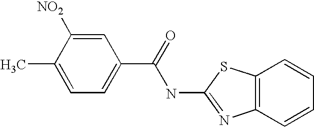 | N-(benzo[d]thiazol-2-yl)-4-methyl- 3-nitrobenzamide |
| 183 | 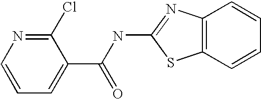 | N-(benzo[d]thiazol-2-yl)-2- chloronicotinamide |
| 184 | 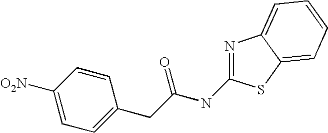 | N-(benzo[d]thiazol-2-yl)-2-(4- nitrophenyl)acetamide |
| 185 | 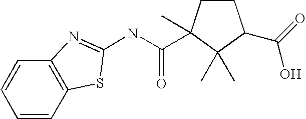 | 3-(benzo[d]thiazol-2- ylcarbamoyl)-2,2,3- trimethylcyclopentanecarboxylic acid |
| 186 | 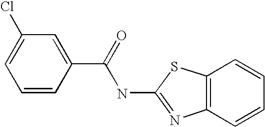 | N-(benzo[d]thiazol-2-yl)-3- chlorobenzamide |
| 187 | 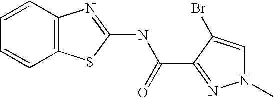 | N-(benzo[d]thiazol-2-yl)-4-bromo- 1-methyl-1H-pyrazole-3- carboxamide |
| 188 | 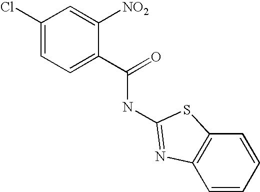 | N-(benzo[d]thiazol-2-yl)-4-chloro- 2-nitrobenzamide |
| 189 | 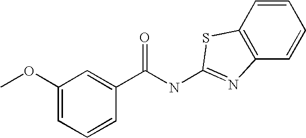 | N-(benzo[d]thiazol-2-yl)-3- methoxybenzamide |
| 190 | 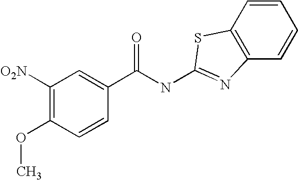 | N-(benzo[d]thiazol-2-yl)-4- methoxy-3-nitrobenzamide |
| 191 | 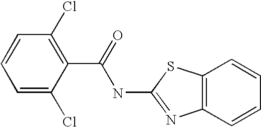 | N-(benzo[d]thiazol-2-yl)-2,6- dichlorobenzamide |
| 192 | 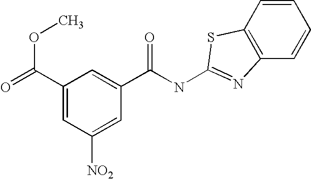 | methyl 3-(benzo[d]thiazol-2- ylcarbamoyl)-5-nitrobenzoate |
| 193 | 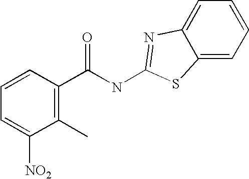 | N-(benzo[d]thiazol-2-yl)-2-methyl- 3-nitrobenzamide |
| 194 | 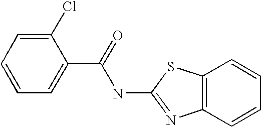 | N-(benzo[d]thiazol-2-yl)-2- chlorobenzamide |
| 195 | 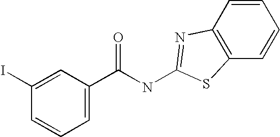 | N-(benzo[d]thiazol-2-yl)-3- iodobenzamide |
| 196 | 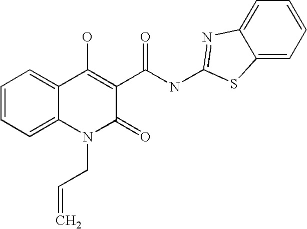 | 1-allyl-N-(benzo[d]thiazol-2-yl)-4- hydroxy-2-oxo-1,2- dihydroquinoiine-3-carboxamide |
| 197 | 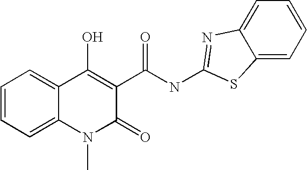 | N-(benzo[d]thiazol-2-yl)-4-hydroxy- 1-methyl-2-oxo-1,2- dihydroquinoline-3-carboxamide |
| 198 | 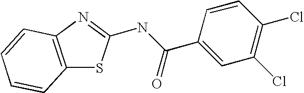 | N-(benzo[d]thiazol-2-yl)-3,4- dichlorobenzamide |
| 199 | 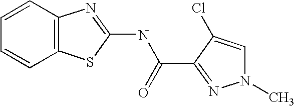 | N-(benzo[d]thiazol-2-yl)-4-chloro- 1-methyl-1H-pyrazole-3- carboxamide |
| 200 | 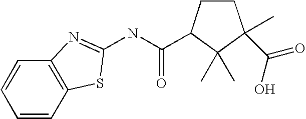 | 3-(benzo[d]thiazol-2- ylcarbamoyl)-1,2,2- trimethylcyclopentanecarboxylic acid |
| 201 | 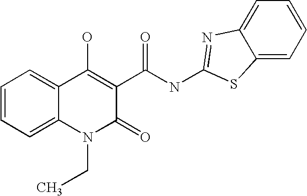 | N-(benzo[d]thiazol-2-yl)-1-ethyl-4- hydroxy-2-oxo-1,2- dihydroquinoline-3-carboxamide |
| 202 | 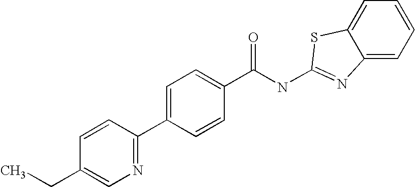 | N-(benzo[b]thiazol-2-yl)-4-(5- ethylpyridin-2-yl)benzamide |
| 203 | 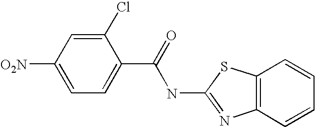 | N-(benzo[b]thiazol-2-yl)-2-chloro- 4-nitrobenzamide |
| 204 | 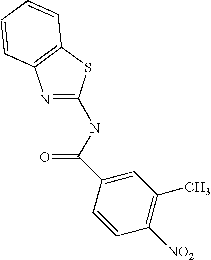 | N-(benzo[b]thiazol-2-yl)-3-methyl- 4-nitrobenzamide |
| 205 | 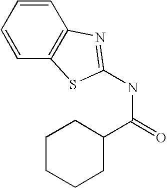 | N-(benzo[d]thiazol-2- yl)cyclohexanecarboxamide |
| 206 | 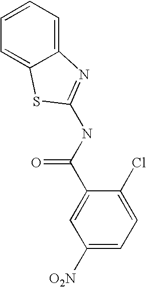 | N-(benzo[b]thiazol-2-yl)-2-chloro- 5-nitrobenzamide |
| 207 | 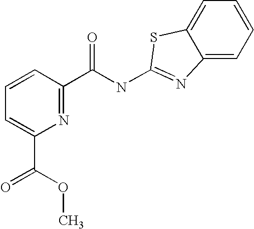 | methyl 6-(benzo[d]thiazol-2- ylcarbamoyl)picolinate |
| 208 | 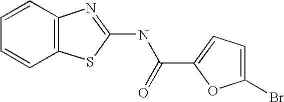 | N-(benzo[b]thiazol-2-yl)-5- bromofuran-2-carboxamide |
| 209 | 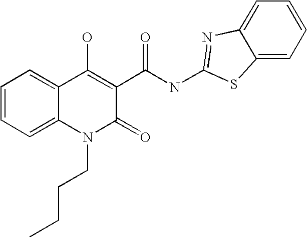 | N-(benzo[d]thiazol-2-yl)-1-butyl-4- hydroxy-2-oxo-1,2- dihydroquinoline-3-carboxamide |
| 210 | 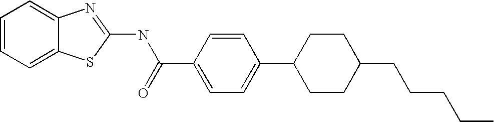 | N-(benzo[d]thiazol-2-yl)-4-(4- pentylcyclohexyl)benzamide |
| 211 | 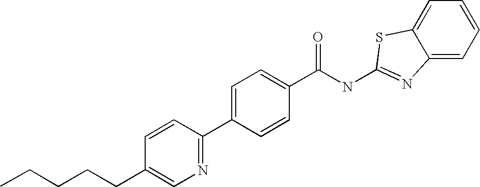 | N-(benzo[d]thiazol-2-yl)-4-(5- pentylpyridin-2-yl)benzamide |
| 212 | 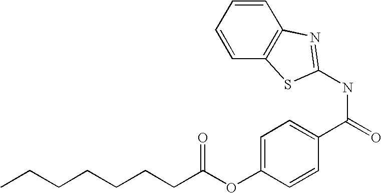 | 4-(benzo[d]thiazol-2- ylcarbamoyl)phenyl octanoate |
| 213 | 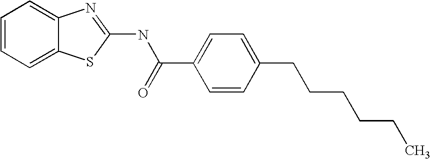 | N-(benzo[d]thiazol-2-yl)-4- hexylbenzamide |
| 214 | 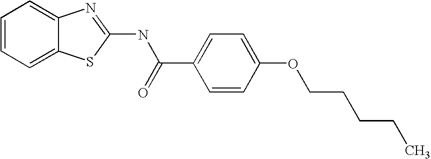 | N-(benzo[d]thiazol-2-yl)-4- (pentyloxy)benzamide |
| 215 | 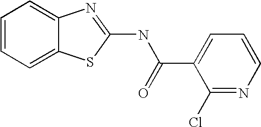 | N-(benzo[d]thiazol-2-yl)-2- chloronicotinamide |
| 216 | 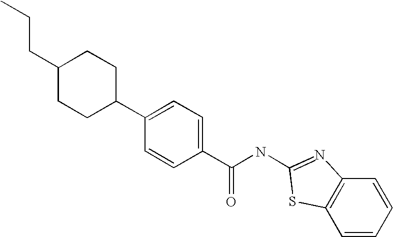 | N-(benzo[d]thiazol-2-yl)-4-(4- propylcyclohexyl)benzamide |
| 217 | 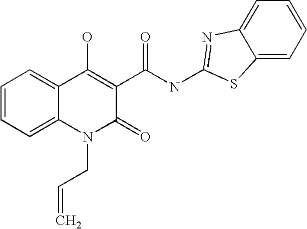 | 1-allyl-N-(benzo[d]thiazol-2-yl)-4- hydroxy-2-oxo-1,2- dihydroquinoline-3-carboxamide |
| 218 | 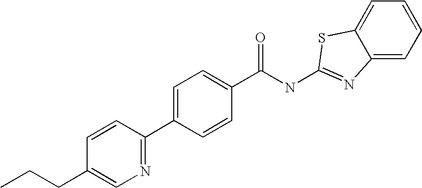 | N-(benzo[d]thiazol-2-yl)-4-(5- propylpyridin-2-yl)benzamide |
| 219 | 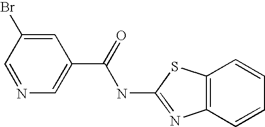 | N-(benzo[d]thiazol-2-yl)-5- bromonicotinamide |
| 220 | 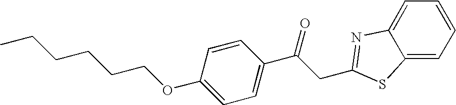 | N-(benzo[d]thiazol-2-yl)-4- (hexyloxy)benzamide |
| 221 | 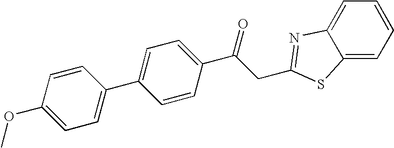 | N-1,3-benzothiazol-2-yl-4′- methoxybiphenyl-4-carboxamide |

TABLE 2

| Cmpd | Structure | Name |
|---|---|---|
| 60 | 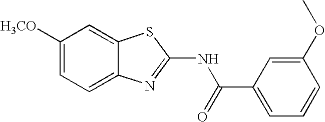 | 3-methoxy-N-(6- methoxybenzo[d]thiazol-2- yl)benzamide |
| 56 | 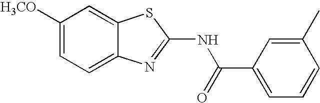 | N-(6-methoxybenzo[d]thiazol-2- yl)-3-methylbenzamide |
| 59 | 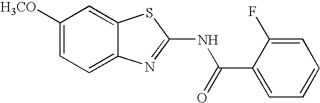 | 2-fluoro-N-(6- methoxybenzo[d]thiazol-2- yl)benzamide |
| 61 | 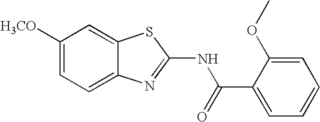 | 2-methoxy-N-(6- methoxybenzo[d]thiazol-2- yl)benzamide |
| 75 | 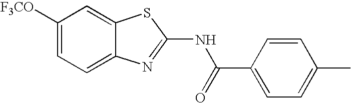 | 4-methyl-N-(6- (trifluoromethoxy)benzo[d]thiazol- 2-yl)benzamide |
| 223 | 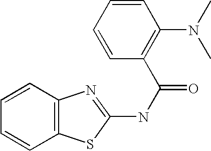 | N-(benzo[d]thiazol-2-yl)-2- (dimethylamino)benzamide |
| 101 | 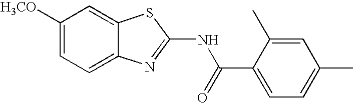 | N-(6-methoxybenzo[d]thiazol-2- yl)-2,4-dimethyibenzamide |
| 222 | 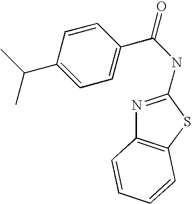 | N-(benzo[d]thiazol-2-yl)-4- isopropylbenzamide |
| 224 | 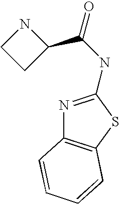 | (R)-N-(benzo[d]thiazol-2- yl)azetidine-2-carboxamide |
| 44 | 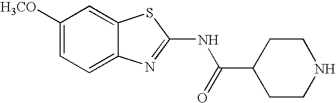 | N-(6-methoxybenzo[d]thiazol-2- yl)piperidine-4-carboxamide |
| 225 | 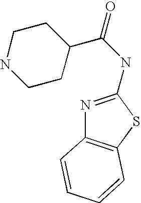 | N-(benzo[d]thiazol-2-yl)piperidine- 4-carboxamide |
| 39 | 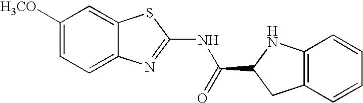 | (R)-N-(6-methoxybenzo[d]thiazol- 2-yl)indoline-2-carboxamide |
| 38 | 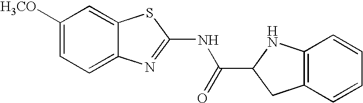 | N-(6-methoxybenzo[d]thiazol-2- yl)indoline-2-carboxamide |
| 47 | 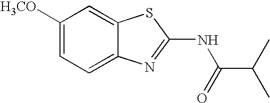 | N-(6-methoxybenzo[d]thiazol-2- yl)isobutyramide |
| 48 | 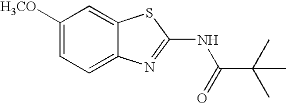 | N-(6-methoxybenzo[d]thiazol-2- yl)pivalamide |
| 46 | 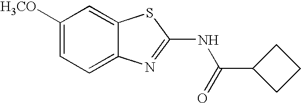 | N-(6-methoxybenzo[d]thiazol-2- yl)cyclobutanecarboxamide |
| 40 | 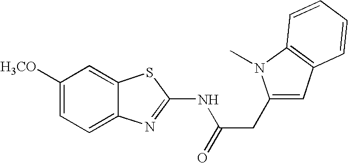 | N-(6-methoxybenzo[d]thiazol-2- yl)-2-(1-methyl-1H-indol-2- yl)acetamide |
| 58 | 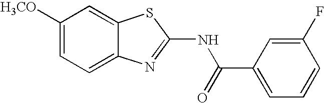 | 3-fluoro-N-(6- methoxybenzo[d]thiazol-2- yl)benzamide |
| 103 | 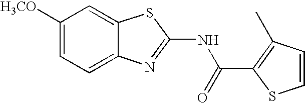 | N-(6-methoxybenzo[d]thiazol-2- yl)-3-methylthiophene-2- carboxamide |
| 104 | 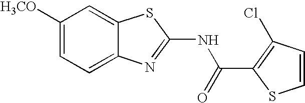 | 3-chloro-N-(6- methoxybenzo[d]thiazol-2- yl)thiophene-2-carboxamide |
| 137 | 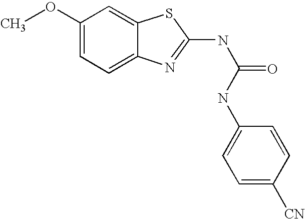 | 1-(4-cyanophenyl)-3-(6- methoxybenzo[d]thiazol-2-yl)urea |
| 138 | 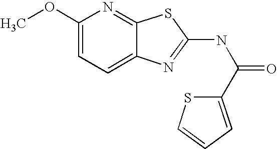 | N-(5-methoxythiazolo[5,4-b]pyridin-2-yl)thiophene-2- carboxamide |
| 140 | 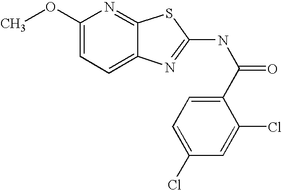 | 2,4-dichloro-N-(5- methoxythiazolo[5,4-b]pyridin-2- yl)benzamide |
| 141 | 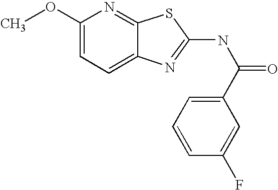 | 3-fluoro-N-(5- methoxythiazolo[5,4-b]pyridin-2- yl)benzamide |
| 142 | 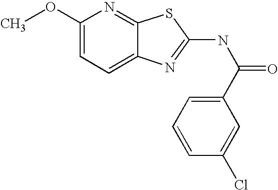 | 3-chloro-N-(5- methoxythiazolo[5,4-b]pyridin-2- yl)benzamide |
| 143 | 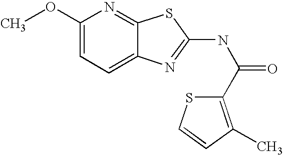 | N-(5-methoxythiazolo[5,4-b]pyridin-2-yl)-3-methylthiophene- 2-carboxamide |
| 144 | 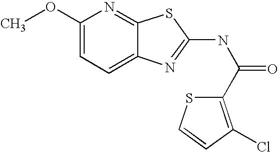 | 3-chloro-N-(5- methoxythiazolo[5,4-b]pyridin-2- yl)thiophene-2-carboxamide |
| 145 | 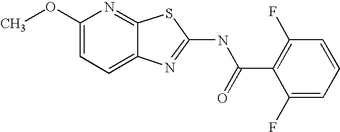 | 2,6-difluoro-N-(5- methoxythiazolo[5,4-b]pyridin-2- yl)benzamide |
| 146 | 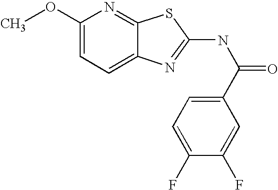 | 3,4-difluoro-N-(5- methoxythiazolo[5,4-b]pyridin-2- yl)benzamide |
| 147 | 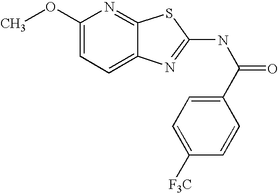 | N-(5-methoxythiazolo[5,4-b]pyridin-2-yl)-4- (trifluoromethyl)benzamide |
| 148 | 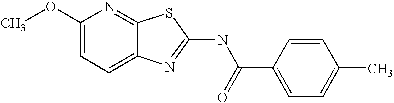 | 4-cyano-N-(5- methoxythiazolo[5,4-b]pyridin-2- yl)benzamide |
| 149 | 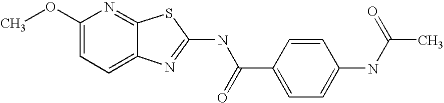 | 4-acetamido-N-(5- methoxythiazolo[5,4-b]pyridin-2- yl)benzamide |
| 150 | 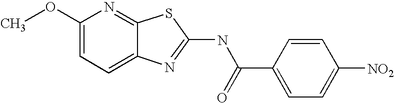 | N-(5-methoxythiazolo[5,4-b]pyridin-2-yl)-4-nitrobenzamide |
| 151 | 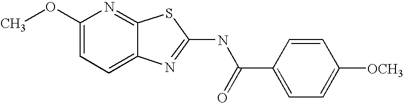 | 4-methoxy-N-(5- methoxythiazolo[5,4-b]pyridin-2- yl)benzamide |
| 153 | 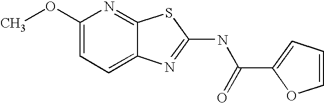 | N-(5-methoxythiazolo[5,4-b]pyridin-2-yl)furan-2- carboxamide |
| 154 | 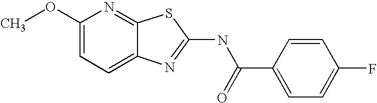 | 4-fluoro-N-(5- methoxythiazolo[5,4-b]pyridin-2- yl)benzamide |
| 226 | 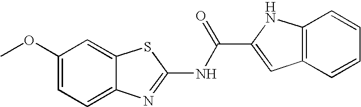 | N-(6-methoxybenzo[d]thiazol-2- yl)-1H-indole-2-carboxamide |

The compounds in the tables above can be prepared using art recognized methods. All of the compounds in this application were named using Chemdraw Ultra version 6.0.2, which is available through Cambridgesoft.co, 100 Cambridge Park Drive, Cambridge, Mass. 02140, Namepro version 5.09, which is available from ACD labs, 90 Adelaide Street West, Toronto, Ontario, M5H, 3V9, Canada, or were derived therefrom.

In a second aspect the invention comprises pharmaceutical compositions comprising a compound of formula (I), (II), (II)-1, (II)-2, (II)-3, (II)-4, (II)-5, (III), (III)-1, (III)-2, (III)-3, (IV), or (V) together with a pharmaceutically acceptable carrier, excipient, or diluent.

The compounds and pharmaceutical compositions of the invention are useful for inhibiting ubiquitination in a cell. Specifically, the pharmaceutical compositions target the E1 activating agent of the ubiquitination process thereby preventing transfer of ATP-activated ubiquitin the E2 conjugating agent. The inhibition of the E1 activating agent prevents ubiquitin of proteins since it also interrupts the downstream function of the E2 conjugating agent and the E3 ligating agent in the ubiquitination pathway. Thus, the pharmaceutical compositions of the invention indirectly inhibit both the E2 conjugating agent and the E3 ligating agent.

Accordingly, the invention also comprises methods of inhibiting ubiquitination in a cell comprising contacting a cell in which inhibition of ubiquitination is desired with a compound or pharmaceutical composition according to the invention. The invention also comprises methods for treating cell proliferative diseases and other conditions in a patient in which ubiquitination is an important component. For example, diseases and conditions that can be treated are cancers and conditions related to cancers. However, any disease and condition in which ubiquitination is a component can be treated with the compounds and pharmaceutical compositions of the invention.

The compounds and compositions of the invention are also useful for preventing and/or treating malaria. Accordingly, the invention further comprises methods of treating and of preventing malaria by administering to a subject (preferably human) an amount of a compound or composition of the invention effective to prevent and/or treat malaria. The invention also provides for the use of a compound or composition of the invention for the manufacture of a medicament for use in treating and/or preventing malaria.

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene.) All atoms are understood to have their normal number of valences for bond formation (i.e., 4 for carbon, 3 for N, 2 for O, and 2, 4, or 6 for S, depending on the oxidation state of the S). On occasion a moiety may be defined, for example, as (A)ₐ-B—, wherein a is 0 or 1. In such instances, when a is 0 the moiety is B— and when a is 1 the moiety is A-B—. Also, a number of moieties disclosed herein exist in multiple tautomeric forms, all of which are intended to be encompassed by any given tautomeric structure. Other stereochemical forms of the compounds of the invention are also encompassed including but not limited to enantiomers, diastereomers, and other isomers such as rotamers.

For simplicity, when a substituent can be of a particular chemical class differing by the number of atoms or groups of the same kind in the moiety (e.g., alkyl, which can be C₁, C₂, C₃, etc.), the number of repeated atoms or groups is represented by a range (e.g., C₁-C₆-alkyl). In such instances each and every number in that range and all sub-ranges are specifically contemplated. Thus, C₁-C₃-alkyl means C₁-, C₂-, C₃-, C₁₋₂, C₁₋₃-, and C₂₋₃-alkyl.

In addition to individual preferred embodiments of each substituent defined herein, the invention also comprises all combinations of preferred substituents.

The term “alkyl” as employed herein refers to straight and branched chain aliphatic groups having from 1 to 12 carbon atoms, preferably 1-8 carbon atoms, more preferably 1-6 carbon atoms, which is optionally substituted with one, two or three substituents. Unless otherwise specified, the alkyl group may be saturated, unsaturated, or partially unsaturated. As used herein, therefore, the term “alkyl” is specifically intended to include alkenyl and alkynyl groups, as well as saturated alkyl groups, unless expressly stated otherwise. Preferred alkyl groups include, without limitation, methyl, ethyl, propyl, isopropyl, butyl, tertbutyl, isobutyl, pentyl, hexyl, vinyl, allyl, isobutenyl, ethynyl, and propynyl.

As employed herein, a “substituted” alkyl, cycloalkyl, aryl, or heterocyclic group is one having between one and about four, preferably between one and about three, more preferably one or two, non-hydrogen substituents. Suitable substituents include, without limitation, halo, hydroxy, nitro, haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups.

The term “cycloalkyl” as employed herein includes saturated and partially unsaturated cyclic hydrocarbon groups having 3 to 12, preferably 3 to 8 carbons, wherein the cycloalkyl group additionally is optionally substituted. Preferred cycloalkyl groups include, without limitation, cyclopropyl, cyclobutyl, cyclopentyl, cyclopentenyl, cyclohexyl, cyclohexenyl, cycloheptyl, cyclooctyl, and adamantyl.

The term “hydrocarbyl” as employed herein includes all alkyl moieties and all cycloalkyl moieties (both as defined above), each alone or in combination. Thus, for example, hydrocarbyl includes methyl, ethyl, propyl, n-butyl, i-butyl, cyclopropyl, cyclohexyl, cyclopropyl-CH₂—, cyclohexyl-(CH₂)₃—, etc.

An “aryl” group is a C₆-C₁₄ aromatic moiety comprising one to three aromatic rings, which is optionally substituted. Preferably, the aryl group is a C₆-C₁₀ aryl group. Preferred aryl groups include, without limitation, phenyl, naphthyl, anthracenyl, and fluorenyl. An “aralkyl” or “arylalkyl” group comprises an aryl group covalently linked to an alkyl group, either of which may independently be optionally substituted or unsubstituted. Preferably, the aralkyl group is C₁-C₆-alkyl-(C₆-C₁₀) aryl, including, without limitation, benzyl, phenethyl, and naphthylmethyl. An “alkaryl” or “alkylaryl” group is an aryl group having one or more alkyl substituents. Examples of alkaryl groups include, without limitation, tolyl, xylyl, mesityl, ethylphenyl, tert-butylphenyl, and methylnaphthyl.

A “heterocyclic” group (or “heterocyclyl”) is a non-aromatic mono-, bi-, or tricyclic structure having from about 3 to about 14 atoms, wherein one or more atoms are selected from the group consisting of N, O, and S. One ring of a bicyclic heterocycle or two rings of a tricyclic heterocycle may be aromatic, as in indan and 9,10-dihydro-anthracene. The heterocyclic group is optionally substituted on carbon with oxo or with one of the substituents listed above. The heterocyclic group may also independently be substituted on nitrogen with alkyl, aryl, aralkyl, alkylcarbonyl, alkylsulfonyl, arylcarbonyl, arylsulfonyl, alkoxycarbonyl, aralkoxycarbonyl, or on sulfur with oxo or lower alkyl. Preferred heterocyclic groups include, without limitation, epoxy, aziridinyl, tetrahydrofuranyl, pyrrolidinyl, piperidinyl, piperazinyl, thiazolidinyl, oxazolidinyl, oxazolidinonyl, and morpholino.

In certain preferred embodiments, the heterocyclic group is a heteroaryl group. As used herein, the term “heteroaryl” refers to groups having 5 to 14 ring atoms, preferably 5, 6, 9, or 10 ring atoms; having 6, 10, or 14π electrons shared in a cyclic array; and having, in addition to carbon atoms, between one and about three heteroatoms selected from the group consisting of N, O, and S. Preferred heteroaryl groups include, without limitation, thienyl, benzothienyl, furyl, benzofuryl, dibenzofuryl, pyrrolyl, imidazolyl, pyrazolyl, pyridyl, pyrazinyl, pyrimidinyl, indolyl, quinolyl, isoquinolyl, quinoxalinyl, tetrazolyl, oxazolyl, thiazolyl, and isoxazolyl.

For simplicity, reference to a “Cₙ-Cₘ” heterocyclyl or “Cₙ-Cₘ” heteroaryl means a heterocyclyl or heteroaryl having from “n” to “m” annular atoms, where “n” and “m” are integers. Thus, for example, a C₅-C₆-heterocyclyl is a 5- or 6-membered ring having at least one heteroatom, and includes pyrrolidinyl (C₅) and piperidinyl (C₆); C₆-heteroaryl includes, for example, pyridyl and pyrimidyl.

In certain other preferred embodiments, the heterocyclic group is fused to an aryl or heteroaryl group. Examples of such fused heterocycles include, without limitation, tetrahydroquinolinyl and dihydrobenzofuranyl.

Additional preferred heterocyclyls and heteroaryls include, but are not limited to, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, 1H-indazolyl, indolenyl, indolinyl, indolizinyl, 3H-indolyl, isobenzofuranyl, isochromanyl, isoindazolyl, isoindolinyl, isoindolyl, isothiazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazolidinyl, pyrazolinyl, pyridazinyl, pyridooxazole, pyridoimidazole, pyridothiazole, pyridinyl, pyrrolidinyl, pyrrolinyl, 2H-pyrrolyl, quinazolinyl, 4H-quinolizinyl, quinuclidinyl, tetrahydroisoquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl, cyclobutenyl and 1,3-dioxoisoindolyl.

A moiety that is substituted is one in which one or more hydrogens have been independently replaced with another chemical substituent. As a non-limiting example, substituted phenyls include 2-fluorophenyl, 3,4-dichlorophenyl, 3-chloro-4-fluoro-phenyl, 2-fluor-3-propylphenyl. As another non-limiting example, substituted n-octyls include 2,4 dimethyl-5-ethyl-octyl and 3-cyclopentyl-octyl. Included within this definition are methylenes (—CH₂—) substituted with oxygen to form carbonyl —CO—).

Unless otherwise stated, as employed herein, when a moiety (e.g., cycloalkyl, hydrocarbyl, aryl, heteroaryl, heterocyclic, urea, etc.) is described as “optionally substituted” it is meant that the group optionally has from one to four, preferably from one to three, more preferably one or two, non-hydrogen substituents. Suitable substituents include, without limitation, halo, hydroxy, oxo (e.g., an annular —CH— substituted with oxo is —C(O)—) nitro, halohydrocarbyl, hydrocarbyl, aryl, aralkyl, alkoxy, aryloxy, amino, acylamino, alkylcarbamoyl, arylcarbamoyl, aminoalkyl, acyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups. Preferred substituents, which are themselves not further substituted (unless expressly stated otherwise) are:
(a) halo, cyano, oxo, carboxy, formyl, nitro, amino, amidino, guanidino,
(b) C₁-C₅ alkyl or alkenyl or arylalkyl imino, carbamoyl, azido, carboxamido, mercapto, hydroxy, hydroxyalkyl, alkylaryl, arylalkyl, C₁-C₈ alkyl, C₁-C₈ alkenyl, C₁-C₈ alkoxy, C₁-C₈ alkoxycarbonyl, aryloxycarbonyl, C₂-C₈ acyl, C₂-C₈ acylamino, C₁-C₈ alkylthio, arylalkylthio, arylthio, C₁-C₈ alkylsulfinyl, arylalkylsulfinyl, arylsulfinyl, C₁-C₈ alkylsulfonyl, arylalkylsulfonyl, arylsulfonyl, C₀-C₆ N-alkyl carbamoyl, C₂-C₁₅ N,N-dialkylcarbamoyl, C₃-C₇ cycloalkyl, aroyl, aryloxy, arylalkyl ether, aryl, aryl fused to a cycloalkyl or heterocycle or another aryl ring, C₃-C₇ heterocycle, C₅-C₁₅ heteroaryl, or any of these rings fused or spiro-fused to a cycloalkyl, heterocyclyl, or aryl, wherein each of the foregoing is further optionally substituted with one more moieties listed in (a), above; and
(c) —(CH₂)ₛ—NR³⁰R³¹, wherein s is from 0 (in which case the nitrogen is directly bonded to the moiety that is substituted) to 6, and R³⁰ and R³¹ are each independently hydrogen, cyano, oxo, carboxamido, amidino, C₁-C₈ hydroxyalkyl, C₁-C₃ alkylaryl, aryl-C₁-C₃ alkyl, C₁-C₈ alkyl, C₁-C₈ alkenyl, C₁-C₈ alkoxy, C₁-C₈ alkoxycarbonyl, aryloxycarbonyl, aryl-C₁-C₃ alkoxycarbonyl, C₂-C₈ acyl, C₁-C₈ alkylsulfonyl, arylalkylsulfonyl, arylsulfonyl, aroyl, aryl, cycloalkyl, heterocyclyl, or heteroaryl, wherein each of the foregoing is further optionally substituted with one more moieties listed in (a), above; or
R³⁰ and R³¹ taken together with the N to which they are attached form a heterocyclyl or heteroaryl, each of which is optionally substituted with from 1 to 3 substituents from (a), above.

The term “halogen” or “halo” as employed herein refers to chlorine, bromine, fluorine, or iodine.

As herein employed, the term “acyl” refers to an alkylcarbonyl or arylcarbonyl substituent.

The term “acylamino” refers to an amide group attached at the nitrogen atom. The term “carbamoyl” refers to an amide group attached at the carbonyl carbon atom. The nitrogen atom of an acylamino or carbamoyl substituent may be additionally substituted. The term “sulfonamido” refers to a sulfonamide substituent attached by either the sulfur or the nitrogen atom. The term “amino” is meant to include NH₂, alkylamino, arylamino, and cyclic amino groups.

### Pharmaceutical Compositions

In a second aspect, the invention provides pharmaceutical compositions comprising an inhibitor of ubiquitination according to the invention and a pharmaceutically acceptable carrier, excipient, or diluent. Compounds of the invention may be formulated by any method well known in the art and may be prepared for administration by any route, including, without limitation, parenteral, oral, sublingual, transdermal, topical, intranasal, intratracheal, or intrarectal. In certain preferred embodiments, compounds of the invention are administered intravenously in a hospital setting. In certain other preferred embodiments, administration may preferably be by the oral route.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, pharmaceutical compositions according to the invention may contain, in addition to the inhibitor, diluents, fillers, salts, buffers, stabilizers, solubilizers, flavors, dyes and other materials well known in the art. The preparation of pharmaceutically acceptable formulations is described in many well known references to one skilled in the art, for example, Remington's Pharmaceutical Sciences, 18th Edition, ed. A. Gennaro, Mack Publishing Co., Easton, Pa., 1990.

As used herein, the term pharmaceutically acceptable salts refers to salts and complexes that retain the desired biological activity of the compounds of the invention and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z-, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate). Moreover, the compounds of the invention can also be administered as prodrugs which can be converted to the active form in vivo.

The active compound is included in the pharmaceutically acceptable carrier or diluent in an amount sufficient to deliver to a patient a therapeutically effective amount without causing serious toxic effects in the patient treated. A preferred dose of the active compound for all of the above-mentioned conditions is in the range from about 0.01 to 500 mg/kg, preferably 0.1 to 100 mg/kg per day, more generally 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

### Inhibition of Ubiquitination

In a third aspect, the invention provides a method of inhibiting ubiquitination in a cell, comprising contacting a cell in which inhibition of ubiquitination is desired with an inhibitor of ubiquitination of the invention.

Measurement of the ubiquitination can be achieved using known methodologies. (See, for example, WO 01/75145, US-2002-0042083-A1 and WO 03/076608, each of which is incorporated by reference in its entirety.)

Preferably, the method according to the third aspect of the invention causes an inhibition of cell proliferation of contacted cells. The phrase “inhibiting cell proliferation” is used to denote an ability of an inhibitor of ubiquitination to retard the growth of cells contacted with the inhibitor as compared to cells not contacted. An assessment of cell proliferation can be made by counting contacted and non-contacted cells using a Coulter Cell Counter (Coulter, Miami, Fla.), photographic analysis with Array Scan II (Cellomics) or a hemacytometer. Where the cells are in a solid growth (e.g., a solid tumor or organ), such an assessment of cell proliferation can be made by measuring the growth with calipers and comparing the size of the growth of contacted cells with non-contacted cells.

Preferably, growth of cells contacted with the inhibitor is retarded by at least 50% as compared to growth of non-contacted cells. More preferably, cell proliferation is inhibited by 100% (i.e., the contacted cells do not increase in number). Most preferably, the phrase “inhibiting cell proliferation” includes a reduction in the number or size of contacted cells, as compared to non-contacted cells. Thus, an inhibitor of ubiquitination according to the invention that inhibits cell proliferation in a contacted cell may induce the contacted cell to undergo growth retardation, to undergo growth arrest, to undergo programmed cell death (i.e., to apoptose), or to undergo necrotic cell death.

In some preferred embodiments, the contacted cell is a neoplastic cell. The term “neoplastic cell” is used to denote a cell that shows aberrant cell growth. Preferably, the aberrant cell growth of a neoplastic cell is increased cell growth. A neoplastic cell may be a hyperplastic cell, a cell that shows a lack of contact inhibition of growth in vitro, a benign tumor cell that is incapable of metastasis in vivo, or a cancer cell that is capable of metastasis in vivo and that may recur after attempted removal. The term “tumorigenesis” is used to denote the induction of cell proliferation that leads to the development of a neoplastic growth. In some embodiments, the ubiquitination inhibitor induces cell differentiation in the contacted cell. Thus, a neoplastic cell, when contacted with an inhibitor of ubiquitination may be induced to differentiate, resulting in the production of a non-neoplastic daughter cell that is phylogenetically more advanced than the contacted cell.

### Treatment for Cell Proliferative Diseases or Conditions

In some preferred embodiments, the contacted cell is in an animal. Thus, in a fourth aspect the invention provides a method for treating a cell proliferative disease or condition in an animal, comprising administering to an animal in need thereof an effective amount of an inhibitor of ubiquitination of the invention. Preferably, the animal is a mammal, more preferably a domesticated mammal. Most preferably, the animal is a human.

The term “cell proliferative disease or condition” is meant to refer to any condition characterized by aberrant cell growth, preferably abnormally increased cellular proliferation. Examples of such cell proliferative diseases or conditions include, but are not limited to, cancer, restenosis, and psoriasis. In particularly preferred embodiments, the invention provides a method for inhibiting neoplastic cell proliferation in an animal comprising administering to an animal having at least one neoplastic cell present in its body a therapeutically effective amount of a ubiquitination inhibitor of the invention. Most preferrably, the invention provides a method for treating cancer comprising administering to a patient in need thereof an effective amount of an inhibitor of ubiquitination of the invention.

The term “therapeutically effective amount” is meant to denote a dosage sufficient to cause inhibition of ubiquitination in the cells of the subject, or a dosage sufficient to inhibit cell proliferation or to induce cell differentiation in the subject. Administration may be by any route, including, without limitation, parenteral, oral, sublingual, transdermal, topical, intranasal, intratracheal, or intrarectal. In certain particularly preferred embodiments, compounds of the invention are administered intravenously in a hospital setting. In certain other preferred embodiments, administration may preferably be by the oral route.

When administered systemically, the ubiquitination inhibitor is preferably administered at a sufficient dosage to attain a blood level of the inhibitor from about 0.01 μM to about 100 μM, more preferably from about 0.05 μM to about 50 μM, still more preferably from about 0.1 μM to about 25 μM, and still yet more preferably from about 0.5 μM to about 20 μM. For localized administration, much lower concentrations than this may be effective, and much higher concentrations may be tolerated. One of skill in the art will appreciate that the dosage of ubiquitination inhibitor necessary to produce a therapeutic effect may vary considerably depending on the tissue, organ, or the particular animal or patient to be treated.

### Treatment of HIV and Related Conditions

In some preferred embodiments, the contacted cell is a cell infected with HIV in a patient. Thus, in a fifth aspect, the invention provides a method for treating HIV infection as well as conditions related to HIV in a patient, comprising administering to a patient in need thereof an effective amount of an inhibitor of ubiquitination of the invention. The preparation, dosage and administration of the inhibitors of ubiquitination of the invention for the treatment of HIV and related conditions can be carried out as described above.

The inhibitors of ubiquitination of the invention are useful for the treatment of HIV infection and related conditions because they can inhibit the replication and spread of HIV. The replication and spread of HIV is decreased by the enzyme APOBEC3G, which acts by causing extensive mutations in the cDNA reverse transcribed from the HIV genomic RNA. This has the effect of terminating the life cycle of HIV. To counteract this effect of APOBEC3G, HIV encodes the protein Vif that functions by decreasing the translation of APOBEC3G and increasing the post-translational degradation of APOBEC3G. The post-translational degradation of APOBEC3G is catalyzed by the 26S proteasome and depends on the polyubiquitination of APOBEC3G. Polyubiquitination serves as a signal for the 26S proteasome to degrade APOBEC3G. Thus, inhibitors of ubiquination of the invention can inhibit the function of the 26S proteasome by prevent the targeting of APOBEC3G to the 26S proteasome so that the intracellular concentration of APOBEC3G is increased. This increased concentration of APOBEC3G in turn inhibits the replication and spread of HIV by diminishing the effect of Vif. The role of APOBEC3G in decreasing HIV replication and spread as well as methods for measuring the activity of the 26S proteasome, APOBEC3G and Vif are described in Stopak et al., “HIV-1 Vif Blocks the Antiviral Activity of APOBEC3G by Impairing Both Its Translation and Intracellular Stability,” Mol. Cell (2003), 12:pp 591-601, which is incorporated by reference in its entirety.

The following examples are intended to further illustrate certain preferred embodiments of the invention, and are not intended to limit the scope of the invention.

### Biological Activity

Biological assays for determining the transfer of ubiquitin from the E1 activating agent to the E2 conjugating agent are described in U.S. patent application Ser. Nos. 09/542,497 and 09/826,312 as well as in the PCT Application WO 01/75145, all of which are incorporated by reference in their entirety. The following assay example illustrates one way by which the ubiquitin ligase inhibitory activity of the compounds of the invention can be assayed. This assay example is not meant to limit in any way the use of the compounds of the invention as ubiquitin ligase inhibitors.

### ASSAY EXAMPLE 1

### E1 to E2 Transfer Assay

The attachment of a ubiquitin moiety to the E2 conjugating agent was assayed using Flag-ubiquitin that was purified from E. coli, E2 Ubch10 that was purified as a His-Ubch10 from E. coli, and E1 that was purified from Sf9 insect cells (Affiniti Research Products, Exeter, U.K.). The wells of a Nickel-substrate 96-well plate (Pierce Chemical) were blocked with 100 μl of 1% casein/phosphate buffered saline (PBS) for 1 hour at room temperature. The blocked Nickel-substrate plate was then washed three times with 200 μl of PBST (0.1% Tween-20 in PBS). Subsequently, Flag-ubiquitin reaction solution was added to each well so that the final concentration was 62.5 mM Tris pH 7.5, 6.25 mg MgCl₂, 0.75 mM DTT, 1.0 μM ATP (low ATP), and 100 ng Flag-ubiquitin. The final reaction solution volume was fixed to 80 μl with Millipore-filtered water. To this was added the following: a ubiquitin agent inhibitor in 10 μl of DMSO, 10 μl of E1 and His-E2 Ubch10 in 20 mM Tris buffer, pH 7.5, and 5% glycerol so that there was 10 ng/well of E1 and 20 ng/well of His-E2 Ubch10. The reaction was then allowed to proceed at room temperature for 1 hour.

After 1 hour, the wells were washed three times with 200 μl of PBST and the amount of E2-ubiquitin complex was measured. For measuring the amount of the E2-ubiquitin complex, 100 μl of Mouse anti-flag diluted 1:10,000 (Sigma Aldrich Fluka Chemicals, St. Louis, Mo.) and anti-mouse HRP diluted 1:15,000 (Jackson Immunoresearch labs, West Grove, Pa.) in PBST were added to each well and allowed to incubate at room temperature for another hour. The wells were then washed three times with 200 μl of PBST and 100 μl of luminol substrate (⅕ dilution) was added. The luminescence of each well was then measured using a fluorimeter to calculate the amount of E2-ubiquitin complex. This procedure was repeated using His-E2 Ubch5C instead of His-E2 Ubch10.

The table below illustrates the inhibitory properties of the pharmaceutical compostions of the invention comprising the compounds listed in the table using the assays described above. Inhibition was measured using IC50 values.

TABLE 3

| Compound | LIGASE_E2-UBCH10 | LIGASE_E2-UBCH5C |
|---|---|---|
| 1 | −+ | −+ |
| 3 | ++ | ++ |
| 4 | ++ | ++ |
| 5 | ++ | ++ |

++ indicates high inhibition; −+ indicates marginal inhibition

### ASSAY EXAMPLE 2

### ATP Competitive Assay

The procedure for carrying out the ATP competitive binding assay was essentially the same as that for the plate binding assay described above with the exception that the concentration of ATP was 200 μM ATP (high ATP).

The table below illustrates the ATP competitive inhibition properties of the pharmaceutical compositions of the invention comprising the compounds listed in the table using the ATP competitive assay described above. Inhibition was measured using IC50 values.

TABLE 4

| | UBC10 | UBC10 | |
|---|---|---|---|
| Compound | 1 μM ATP | 200 μM ATP | Results |
| 3 | ++ | −− | ATP competitve |
| 4 | ++ | −− | ATP competitve |
| 5 | ++ | −− | ATP competitve |
| 14 | ++ | − | ATP competitve |

++ indicates high inhibition; −+ indicates marginal inhibition

Table 5 also shows ATP inhibition properties for additional compounds described herein. Inhibition was measured using IC50 values.

TABLE 5

| Cmpd | UBC10 |
|---|---|
| 15 | ++ |
| 16 | ++ |
| 17 | ++ |
| 18 | −− |
| 19 | ++ |
| 20 | −− |
| 21 | ++ |
| 22 | ++ |
| 23 | −− |
| 24 | ++ |
| 25 | −− |
| 26 | ++ |
| 27 | ++ |
| 28 | ++ |
| 29 | ++ |
| 30 | ++ |
| 31 | ++ |
| 32 | ++ |
| 33 | ++ |
| 34 | ++ |
| 35 | −− |
| 36 | ++ |
| 37 | −− |
| 38 | ++ |
| 39 | ++ |
| 40 | ++ |
| 41 | ++ |
| 42 | ++ |
| 43 | −− |
| 44 | −− |
| 45 | ++ |
| 46 | ++ |
| 47 | −− |
| 48 | ++ |
| 49 | ++ |
| 50 | −− |
| 51 | ++ |
| 52 | −− |
| 53 | ++ |
| 54 | −− |
| 55 | −− |
| 56 | ++ |
| 57 | ++ |
| 58 | ++ |
| 59 | ++ |
| 60 | ++ |
| 61 | −− |
| 62 | −− |
| 63 | −− |
| 64 | −− |
| 65 | −− |
| 66 | −− |
| 67 | −− |
| 68 | −− |
| 69 | −− |
| 70 | −− |
| 71 | −− |
| 72 | −− |
| 73 | −− |
| 74 | −− |
| 75 | −− |
| 76 | −− |
| 77 | ++ |
| 78 | −− |
| 79 | ++ |
| 80 | −− |
| 81 | ++ |
| 82 | ++ |
| 83 | −− |
| 84 | ++ |
| 85 | ++ |
| 86 | −− |
| 87 | ++ |
| 88 | −− |
| 89 | −− |
| 90 | ++ |
| 91 | −− |
| 92 | −− |
| 93 | −− |
| 94 | −− |
| 95 | ++ |
| 96 | ++ |
| 97 | ++ |
| 98 | −− |
| 99 | −− |
| 100 | ++ |
| 101 | ++ |
| 103 | ++ |
| 104 | −− |
| 109 | −− |
| 113 | |
| 120 | ++ |
| 124 | −− |
| 126 | −− |
| 132 | −− |
| 136 | −− |
| 137 | −− |
| 138 | ++ |
| 139 | −− |
| 140 | −− |
| 141 | ++ |
| 142 | ++ |
| 144 | −− |
| 145 | −− |
| 146 | ++ |
| 147 | −− |
| 148 | −− |
| 149 | ++ |
| 150 | ++ |
| 151 | ++ |
| 152 | ++ |
| 153 | ++ |
| 154 | ++ |
| 155 | ++ |
| 156 | −− |
| 159 | ++ |
| 164 | −− |
| 168 | −− |
| 169 | −− |
| 170 | −− |
| 172 | −− |
| 174 | −− |
| 176 | −− |
| 181 | −− |
| 186 | −− |
| 189 | −− |
| 194 | −− |
| 195 | ++ |
| 205 | −− |
| 208 | −− |
| 222 | −− |
| 223 | −− |
| 224 | −− |
| 225 | −− |

++ indicates inhibition at 50 μM or or less ; −− indicates marginal or no inhibition detected with this assay

### General Synthetic Procedure

The compounds of the invention can be prepared using general synthetic procedures. The starting components are readily prepared from benzene and phenols to which any kind of substitutions can be made according to procedures well known to those skilled in the art and commercially available. Many of the compounds are available commercially.

The compounds of the invention can be prepared according to Scheme 1. The amine 1a is reacted with the acyl chloride 2a to produce the 2-substituted benzothiazole 3a. One skilled in the art would recognize that to obtain compounds with a variety of groups attached at the 2-position of the benzothiazole, the benzoyl chloride 2a can be replaced with any suitable acyl chloride. Similarly, replacing the amine 1a with any suitable amine, for example, 2-amino-indole or 2-aminobenzoimidazole, the corresponding 2-substituted indole or 2-substituted benzoimidazole can be obtained. Scheme 1 is only one way to prepare the compounds of the invention and is not meant to be limiting in any way.

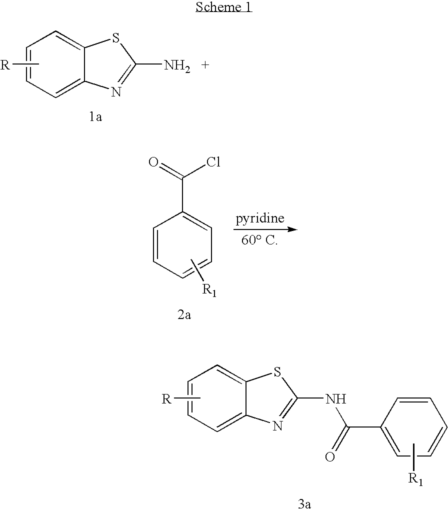

### CHEMISTRY EXAMPLES

### N-(5-methoxythiazolo[5,4-b]pyridin-2-yl)thiophene-2-carboxamide

A solution of 2-Amino-5-methoxythiazolo[5,4-b]pyridine (45 mg, 0.25 mmol) and 2-thiophenecarbonyl chloride (53 mL, 0.50 mmol) in pyridine was heated at 100 C overnight. The reaction mixture was cooled, diluted with ethyl acetate and rinsed with brine. The solution was dried over MgSO₄, eluted through a small silica column (1:1 ethyl acetate:hexanes), and concentrated in vacuo. The residue was purified by preparative HPLC.

¹H NMR (DMSO-d₆, 300 MHz) δ 8.27 (br d, J=3.3 Hz, 1H), 8.04 (d, J=8.7 Hz, 1H), 7.99 (dd, J=1.2, 12.3 Hz, 1H), 7.26 (dd, J=3.6, 4.8 Hz, 1H), 6.92 (d, J=8.7 Hz, 1H), 3.91 (s, 3H).

LCMS purity 100%. MS Found 292 (MH⁺).

### 2,4-dichloro-N-(5-methoxythiazolo[5,4-b]pyridin-2-yl)benzamide

¹H NMR (DMSO-d₆, 300 MHz) δ 8.07 (d, J=8.7 Hz, 1H), 7.79 (m, 1H), 7.72 (d, J=8.4 Hz, 1H), 7.57 (dd, J=2.1, 8.4 Hz, 1H), 6.93 (d, J=8.7 Hz, 1H), 3.93 (s, 3H).

LCMS purity 100%. MS Found 354 (MH⁺).

### 3-fluoro-N-(5-methoxythiazolo[5,4-b]pyridin-2-yl)benzamide

¹H NMR (DMSO-d₆, 300 MHz) δ 8.07 (d, J=8.7 Hz, 1H), 7.97-7.91 (m, 2H), 7.65-7.47 (m, 2H), 6.93 (d, J=4.8 Hz, 1H), 3.93 (s, 3H).

LCMS purity 100%. MS Found 304 (MH⁺).

### 3-chloro-N-(5-methoxythiazolo[5,4-b]pyridin-2-yl)benzamide

¹H NMR (DMSO-d₆, 300 MHz) δ 8.16 (m, 1H), 8.08-8.03 (m, 2H), 7.71 (br d, J=7.8 Hz, 1H), 7.57 (t, J=7.8 Hz, 1H), 6.92 (d, J=8.4 Hz, 1H), 3.92 (s, 3H).

LCMS purity 100%. MS Found 320 (MH⁺).

### N-(5-methoxythiazolo[5,4-b]pyridin-2-yl)-3-methylthiophene-2-carboxamide

¹H NMR (DMSO-d₆, 300 MHz) δ 7.98 (br d, J=8.7 Hz, 1H), 7.76 (d, J=4.8 Hz, 1H), 7.06 (d, J=4.8 Hz, 1H), 6.91 (d, J=8.7 Hz, 1H), 3.91 (s, 3H), 2.52 (s, 3H).

LCMS purity 100%. MS Found 306 (MH⁺).

### 3-chloro-N-(5-methoxythiazolo[5,4-b]pyridin-2-yl)thiophene-2-carboxamide

¹H NMR (DMSO-d₆, 300 MHz) δ 7.97 (br d, J=5.1 Hz, 2H), 7.24 (d, J=5.1 Hz, 1H), 6.93 (d, J=8.7 Hz, 1H), 3.92 (s, 3H).

LCMS purity 100%. MS Found 326 (MH⁺).

### 2,6-difluoro-N-(5-methoxythiazolo[5,4-b]pyridin-2-yl)benzamide

¹H NMR (DMSO-d₆, 300 MHz) δ 8.08 (d, J=9 Hz, 1H), 7.69-7.59 (m, 1H), 7.27 (t, J=8.4 Hz, 2H), 6.94 (d, J=8.4 Hz, 1H), 3.93 (s, 3H).

LCMS purity 100%. MS Found 322 (MH⁺).

### 3,4-difluoro-N-(5-methoxythiazolo[5,4-b]pyridin-2-yl)benzamide

¹H NMR (DMSO-d₆, 300 MHz) δ 8.22-8.15 (m, 1H), 8.06 (d, J=8.7 Hz, 1H), 8.02-7.98 (m, 1H), 7.68-7.59 (m, 1H), 6.93 (d, J=8.4 Hz, 1H), 3.92 (s, 3H).

LCMS purity 100%. MS Found 322 (MH⁺).

### N-(5-methoxythiazolo[5,4-b]pyridin-2-yl)-4-(trifluoromethyl)benzamide

¹H NMR (DMSO-d₆, 300 MHz) δ 8.27 (d, J=8.4 Hz, 2H), 8.07 (d, J=8.7 Hz, 1H), 7.92 (d, J=8.7 Hz, 2H), 6.94 (d, J=9 Hz, 1H), 3.93 (s, 3H).

LCMS purity 100%. MS Found 354 (MH⁺).

### 4-cyano-N-(5-methoxythiazolo[5,4-b]pyridin-2-yl)benzamide

¹H NMR (DMSO-d₆, 300 MHz) δ 8.22 (d, J=8.4 Hz, 2H), 8.08 (d, J=8.7 Hz, 1H), 8.04 (d, J=8.4 Hz, 1H), 6.94 (d, J=8.7 Hz, 1H), 3.93 (s, 3H).

LCMS purity 100%. MS Found 311 (MH⁺).

### 4-acetamido-N-(5-methoxythiazolo[5,4-b]pyridin-2-yl)benzamide

¹H NMR (DMSO-d₆, 300 MHz) δ 10.27 (s, 1H), 8.06 (t, J=8.7 Hz, 3H), 7.72 (d, J=9 Hz, 2H), 6.91 (d, J=8.7 Hz, 1H), 3.92 (s, 3H), 2.09 (s, 3H).

LCMS purity 100%. MS Found 343 (MH⁺).

### N-(5-methoxythiazolo[5,4-b]pyridin-2-yl)-4-nitrobenzamide

¹H NMR (DMSO-d₆, 300 MHz) δ 8.38-8.29 (m, 4H), 8.08 (d, J=8.7 Hz, 1H), 6.95 (d, J=9.6 Hz), 3.94 (s, 3H).

LCMS purity 100%. MS Found 331 (MH⁺).

### 4-methoxy-N-(5-methoxythiazolo[5,4-b]pyridin-2-yl)benzamide

¹H NMR (DMSO-d₆, 300 MHz) δ 8.11 (d, J=9 Hz, 2H), 8.04 (d, J=8.4 Hz, 1H), 7.08 (d, J=9.3 Hz, 2H), 6.91 (d, J=8.7 Hz, 1H), 3.92 (s, 3H), 3.85 (s, 3H).

LCMS purity 100%. MS Found 316 (MH⁺).

### N-(5-methoxythiazolo[5,4-b]pyridin-2-yl)furan-2-carboxamide

¹H NMR (DMSO-d₆, 300 MHz) δ 8.05-8.02 (m, 2H), 7.70 (d, J=3.6 Hz, 1H), 6.91 (d, J=8.7 Hz, 1H), 6.75-6.74 (m, 1H), 3.91 (s, 3H).

LCMS purity 100%. MS Found 276 (MH⁺).

### 4-fluoro-N-(5-methoxythiazolo[5,4-b]pyridin-2-yl)benzamide

¹H NMR (DMSO-d₆, 300 MHz) δ 8.20-8.16 (m, 2H), 8.05 (d, J=8.7 Hz, 1H), 7.39 (t, J=8.7 Hz, 1H), 6.92 (d, J=8.7 Hz, 1H), 3.92 (s, 3H).

LCMS purity 100%. MS Found 304 (MH⁺).

### N-(6-methoxybenzo[d]thiazol-2-yl)-1H-indole-2-carboxamide

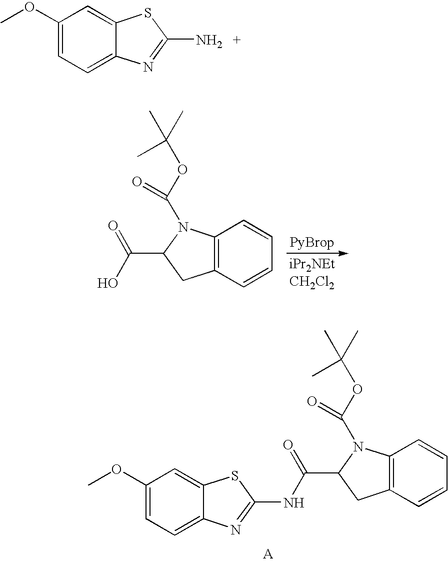

Compound A. A solution of 2-amino-6-methoxybenzothiazole (100 mg, 0.6 mmol), 1-[(tert-butyl)oxycarbonyl]-(±)-indoline-2-carboxylic acid (237 mg, 0.9 mmol), bromo-tris-pyrrolidino-phosphonium hexafluorophosphate (468 mg, 0.9 mmol), and N,N-diisopropylethylamine (300 μL, 1.8 mmol) was prepared at room temperature and allowed to stir over night. The reaction mixture was diluted with CH₂Cl₂, and rinsed with saturated citric acid, and brine. The organic fraction was dried over MgSO₄, filtered, and concentrated. The residue was purified by silica gel chromatography (1:4 to 1:1 ethyl acetate:hexanes) to afford product (A) as a white solid (210 mg, 82%) which was pure by LCMS analysis.

LCMS purity 100%. MS Found 426 (MH⁺), 326 (MH⁺—BOC)

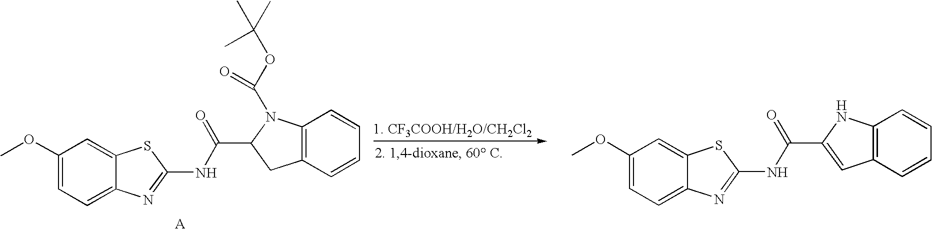

A sample of A (100 mg, 0.235 mmol) was treated with a solution of trifluoroacetic acid (3 mL), CH₂Cl₂ (300 uL), and H₂O (100 uL) at room temperature for 5 hours. The reaction mixture was concentrated in vacuo and used for the next step without purification. The crude reaction mixture was dissolved in 1,4-dioxane (3 mL) and allowed to stir at 60 C for 4 days. The reaction mixture was concentrated in vacuo and the residue purified by silica gel chromatography (1:4 to 1:2 ethyl acetate:hexanes) to afford N-(6-methoxybenzo[d]thiazol-2-yl)-1H-indole-2-carboxamide as a light yellow solid (51 mg, 67% yield).

¹H NMR (CDCl₃, 300 MHz) δ 11.91 (br s, 1H), 7.68-7.67 (m, 2H), 7.65 (d, J=3 Hz, 1H), 7.60 (d, J=2.7 Hz, 1H), 7.46 (d, J=8 Hz, 1H), 7.25 (t, J=6.9 Hz, 1H), 7.09-7.02 (m, 2H), 3.81 (s, 3H).

LCMS purity 100%. MS Found 324 (MH⁺).

The invention has been described with reference to various specific and preferred embodiments and techniques. However, it should be understood that many variations and modifications may be made while remaining within the spirit and scope of the invention.

1.-28. (canceled)

29. A compound of the formula (III): 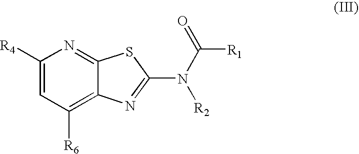
or a pharmaceutically acceptable salt thereof, wherein
R₁ is C₁-C₆ alkyl, aryl, heteroaryl, cycloalkyl, heterocyclyl, -aryl-W-aryl, -aryl-W-heterocyclyl, or heterocyclyl-W-aryl, wherein W is a bond, —O—, —SO₂—, or —C(═O)—;
R₂ is H, C₁-C₆ alkyl, or is linked to a carbon of R₁ through a carbonyl group;
R₄ and R₆ are independently H, halogen, C(O)R₇, NR₈R₉, nitro, C₁₋₆-alkyl, C₁₋₆-alkoxy, OCF₃, CF₃, aryl, —C₁₋₆-alkyl-aryl, heteroaryl, —C₁₋₁₆-alkyl-heteroaryl, C(0)NR₈R₉, C(0)C(O)NR₈R₉, C₁-C₆ alkyl-C(O)—NH—, NR₈R₉—SO₂— or R₁₀—S0₂—;
R₇ is hydrogen, C₁₋₆-alkyl, C₁₋₆-alkoxy, C(Z)-R₁₁ where Z is CH₂ or O, heteroaryl, aryl, or a group of the formula 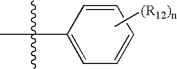
wherein n is 1 to 5 and each R₁₂ is the same or different and is C₁₋₆-alkyl, hydroxy, halogen, nitro, oxo, amino, halo-C₁₋₆-alkyl, C₁₋₆-alkoxy, halo-C₁₋₆-alkoxy, or cyano, NHC(O)—C₁₋₆-alkyl, NHC(O)—C₂₋₆-alkylene, C(O)—O—C₁₋₆-alkyl, or C(O)-aryl;
R₈ and R₉ are independently hydrogen, or C₁-C₆-alkyl;
R₁₀ is C₁₋₆-alkyl, C₁₋₆-alkyl-aryl, aryl, or heteroaryl;
R₁₁ is C₁₋₆-alkyl, C₁₋₆-alkyl-aryl, aryl, or NR₈R₉;
with the proviso that R₄ and R₆ are not simultaneously hydrogen; and
wherein each one of the alkyl, aryl, heteroaryl, or heterocyclyl of the above groups is optionally substituted with one or more groups selected from C₁₋₈-alkyl, C₂-C₆ alkenyl, hydroxy, halogen, nitro, oxo, amino, monoalkylamino, dialkylamino, halo-C₁₋₈-alkyl, C₁₋₈-alkoxy, halo-C₁₋₈-alkoxy, cyano, NHC(O)—C₁₋₈-alkyl, NHC(O)-cycloalkyl, NHC(O)—C₂₋₆-alkenyl, NHC(O)-aryl-C(O)—O—C₁₋₈-alkyl, C(O)—O—R₁₃, —O—C(O)—C₁-C₈ alkyl, or C(O)-aryl, wherein R₁₃ is H or C₁-C₈ alkyl,
and two substituents on aryl, together with the atoms to which they are attached, optionally form a dioxane ring;
provided that the compound is not: N-(5-methoxythiazolo[5,4-b]pyridin-2-yl)benzamide; N-(5-methoxythiazolo[5,4-b]pyridin-2-yl)-4-methylbenzamide; or 4-chloro-N-(5-methoxythiazolo[5,4-b]pyridin-2-yl)benzamide.

30. The compound of claim 29 wherein R₁ is aryl.

31. The compound of claim 30 wherein R₁ is phenyl, optionally substituted with 1, 2, or 3 groups independently selected from halogen, halo-C₁-C₆ alkyl, cyano, —N—C(O)—C₁-C₆ alkyl, nitro, C₁-C₆ alkoxy, and C₁-C₆ alkyl.

32. The compound of claim 29 wherein R₁ is furanyl or thiophene, which are optionally substituted with 1, 2, or 3 groups independently selected from halogen, halo-C₁-C₆ alkyl, cyano, —N—C(O)—C₁-C₆ alkyl, nitro, C₁-C₆ alkoxy, and C₁-C₆ alkyl.

33. The compound of claim 31 wherein R₆ is hydrogen, and R₄ is C₁₋₆-alkoxy.

34. The compound of claim 32 wherein R₆ is hydrogen, and R₄ is C₁₋₆-alkoxy.

35. A compound selected from the group consisting of:
N-(5-methoxythiazolo[5,4-b]pyridin-2-yl)thiophene-2-carboxamide;
2,4-dichloro-N-(5-methoxythiazolo[5,4-b]pyridin-2-yl)benzamide;
3-fluoro-N-(5-methoxythiazolo[5,4-b]pyridin-2-yl)benzamide;
3-chloro-N-(5-methoxythiazolo[5,4-b]pyridin-2-yl)benzamide;
N-(5-methoxythiazolo[5,4-b]pyridin-2-yl)-3-methylthiophene-2-carboxamide;
3-chloro-N-(5-methoxythiazolo[5,4-b]pyridin-2-yl)thiophene-2-carboxamide;
2,6-difluoro-N-(5-methoxythiazolo[5,4-b]pyridin-2-yl)benzamide;
3,4-difluoro-N-(5-methoxythiazolo[5,4-b]pyridin-2-yl)benzamide;
N-(5-methoxythiazolo[5,4-b]pyridin-2-yl)-4-(trifluoromethyl)benzamide;
4-cyano-N-(5-methoxythiazolo[5,4-b]pyridin-2-yl)benzamide;
4-acetamido-N-(5-methoxythiazolo[5,4-b]pyridin-2-yl)benzamide;
N-(5-methoxythiazolo[5,4-b]pyridin-2-yl)-4-nitrobenzamide;
4-methoxy-N-(5-methoxythiazolo[5,4-b]pyridin-2-yl)benzamide;
N-(5-methoxythiazolo[5,4-b]pyridin-2-yl)furan-2-carboxamide;
4-fluoro-N-(5-methoxythiazolo[5,4-b]pyridin-2-yl)benzamide; and
pharmaceutically acceptable salts thereof.
